(19) United States
(12) Patent Application Publication
(10) Pub. No.: US 2024/0336586 A1
(43) Pub. Date: Oct. 10, 2024

(54) METHODS FOR SYNTHESIS OF RACEMIC, SCALEMIC, AND CHIRAL ALPHA-TOCOTRIENOL QUINONE

(71) Applicant: PTC Therapeutics, Inc., South Plainfield, NJ (US)

(72) Inventors: Peter GIANNOUSIS, Pacifica, CA (US); Paul MOLLARD, San Jose, CA (US); Sebastien GOUDEDRANCHE, Sierentz (FR); Tomoya KARASAWA, Osaka (JP)

(21) Appl. No.: 18/293,122

(22) PCT Filed: Jul. 27, 2022
(86) PCT No.: PCT/US2022/038501

§ 371 Date: Jan. 29, 2024

Related U.S. Application Data
(60) Provisional application No. 63/227,221, filed on Jul. 29, 2021.

(51) Int. Cl. C07D 309/10; C07C 50/28; C07D 303/04

(52) U.S. Cl. CPC C07D 309/10; C07C 50/28; C07D 303/04

(57) ABSTRACT

The application discloses methods useful for the synthesis of racemic, scalemic, and chiral alpha-tocotrienol quinone, including synthetic intermediates and methods for making said synthetic intermediates.

This application claims priority to, and the benefit of, U.S. Ser. No. 63/227,221 filed Jul. 29, 2021, the entirety of which is hereby incorporated by reference for all purposes.

### TECHNICAL FIELD

The application discloses methods useful for the synthesis of racemic, scalemic, and chiral alpha-tocotrienol quinone. Further provided are synthetic intermediates useful in the synthesis of racemic, scalemic, and chiral alpha-tocotrienol quinone, as well as methods for making said synthetic intermediates.

### BACKGROUND

Methods relating to the synthesis of alpha-tocotrienol quinone are described in PCT/US09/62212, PCT/US 12/047455, and PCT/US2016/067377.

What is needed are methods for the synthesis of racemic, scalemic, and chiral alpha-tocotrienol quinone that do not rely on a natural plant material, such as palm oil, as the starting material. What is further needed are methods for making synthetic intermediates useful in the synthesis of racemic, scalemic, and chiral alpha-tocotrienol quinone.

### BRIEF SUMMARY

In one aspect is a compound of the formula:

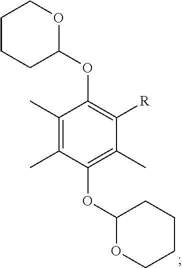

wherein R is —CH₃ or

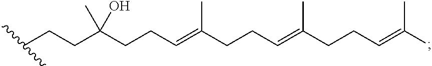

or a salt thereof; and/or an isomer thereof;
and/or a solvate or a hydrate thereof.
   In one embodiment is a compound wherein R is:

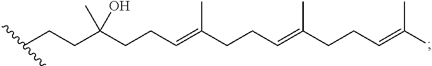

or a salt thereof; and/or an isomer thereof; and/or a solvate or a hydrate thereof. The compound may be present as any single isomer thereof, or a combination of two or more isomers, including any combination of E/Z and R/S isomers. In some embodiments, the compound is wherein R is:

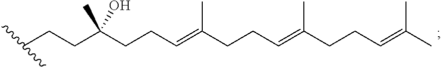 [SMILES: *CC[C@](C)(O)CC/C=C(\C)CC/C=C(\C)CCC=C(C)C]

or a salt thereof; and/or a solvate or a hydrate thereof. In some embodiments, the compound is wherein R is:

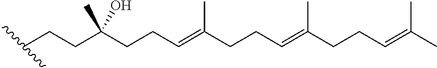 [SMILES: *CC[C@](C)(O)CC/C=C(\C)CC/C=C(\C)CCC=C(C)C]

and the compound is the E-isomer at the 6 and 10-positions: or a salt thereof; and/or a solvate or a hydrate thereof. In some embodiments, including any of the foregoing embodiments, the salt is a lithium salt. In some embodiments, including any of the foregoing embodiments, the compound is not a salt. In some embodiments, including any of the foregoing embodiments, the compound is not a solvate or hydrate. In another embodiment is a compound wherein R is —CH₃; or a salt thereof; and/or an isomer thereof; and/or a solvate or a hydrate thereof. The compound may be present as any single isomer thereof, or a combination of two or more isomers, including any combination of R/S isomers. In some embodiments, including any of the foregoing embodiments, the salt is a lithium salt. In some embodiments, including any of the foregoing embodiments, the compound is not a salt. In some embodiments, including any of the foregoing embodiments, the compound is not a solvate or hydrate.

In another aspect is a compound of the formula:

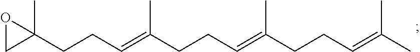 [SMILES: CC(C)=CCC/C(C)=C/CC/C(C)=C/CCC1(C)CO1]

or a salt thereof; and/or an isomer thereof; and/or a solvate or hydrate thereof. The compound may be present as any single isomer thereof, or a combination of two or more isomers, including any combination of E/Z and R/S isomers. In some embodiments, the compound has the formula:

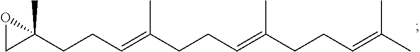 [SMILES: CC(C)=CCC/C(C)=C/CC/C(C)=C/CC[C@]1(C)CO1]

or a salt thereof; and/or a solvate or hydrate thereof. In some embodiments, the compound has the formula:

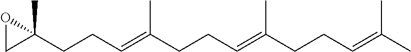 [SMILES: CC(C)=CCC/C(C)=C/CC/C(C)=C/CC[C@]1(C)CO1]

and the compound is the E-isomer at the 3 and 7-positions; or a salt thereof; and/or a solvate or a hydrate thereof.

In another aspect is a method of making the compound

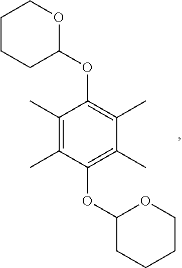 [SMILES: Cc1c(C)c(OC2CCCCO2)c(C)c(C)c1OC1CCCCO1]

or a salt thereof; and/or an isomer thereof; and/or a solvate or a hydrate thereof, comprising: contacting

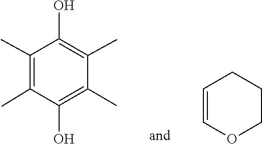

in the presence of a sulfonic acid. In some embodiments, the sulfonic acid is selected from the group consisting of: methanesulfonic, benzenesulfonic, and para-toluenesulfonic acid. In some embodiments, the sulfonic acid is para-toluenesulfonic acid hydrate. In some embodiments, including any of the foregoing embodiments, the reaction is performed in MeTHF. In some embodiments, including any of the foregoing embodiments, the reaction is performed at a temperature between about 20 to about 30° C., inclusive. In some embodiments, including any of the foregoing embodiments, the reaction is performed for at least about 12 hours. In some embodiments, including any of the foregoing embodiments, the product is triturated in a C₁-C₁₀ alkane. In some embodiments, including any of the foregoing embodiments, the C₁-C₁₀ alkane is selected from the group consisting of: pentanes, hexanes, heptanes, and cyclohexane. In some embodiments, including any of the foregoing embodiments, the C₁-C₁₀ alkane is heptanes.

In another aspect is a method of making the compound:

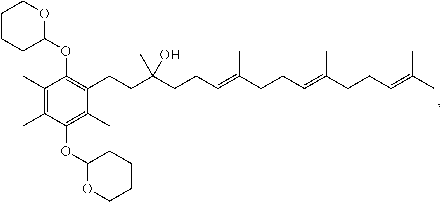 [SMILES: CC(C)=CCC/C(C)=C/CC/C(C)=C/CCC(C)(O)CCc1c(C)c(OC2CCCCO2)c(C)c(C)c1OC1CCCCO1]

or a salt thereof; and/or an isomer thereof; and/or a solvate or a hydrate thereof, comprising: (a) contacting the compound:

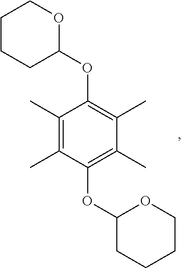 [SMILES: Cc1c(C)c(OC2CCCCO2)c(C)c(C)c1OC1CCCCO1]

or a salt thereof; and/or an isomer thereof; and/or a solvate or a hydrate thereof, with an alkyl lithium salt, followed by (b) adding the compound:

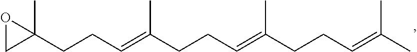 [SMILES: CC(C)=CCC/C(C)=C/CC/C(C)=C/CCC1(C)CO1]

or a salt thereof; and/or an isomer thereof; and/or a solvate or hydrate thereof. In some embodiments, including any of the foregoing embodiments, the alkyl lithium salt is selected from the group consisting of C₁-C₁₀ alkyl lithium salts. In some embodiments, including any of the foregoing embodiments, the alkyl lithium salt is selected from the group consisting of C₄-C₇ alkyl lithium salts. In some embodiments, including any of the foregoing embodiments, the alkyl lithium salt is n-BuLi. In some embodiments, including any of the foregoing embodiments, the compound

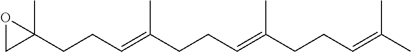 [SMILES: CC(C)=CCC/C(C)=C/CC/C(C)=C/CCC1(C)CO1]

is

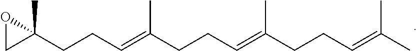 [SMILES: CC(C)=CCC/C(C)=C/CC/C(C)=C/CC[C@]1(C)CO1]

In some embodiments, including any of the foregoing embodiments, the compound

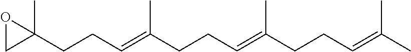 [SMILES: CC(C)=CCC/C(C)=C/CC/C(C)=C/CCC1(C)CO1]

is

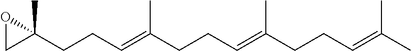 [SMILES: CC(C)=CCC/C(C)=C/CC/C(C)=C/CC[C@]1(C)CO1]

and the compound is the E-isomer at the 3 and 7-positions. In some embodiments, including any of the foregoing embodiments, the reaction in (a) is performed in a non-coordinating solvent. In some embodiments, including any of the foregoing embodiments, the non-coordinating solvent is selected from the group consisting of C₁-C₁₀ alkanes. In some embodiments, including any of the foregoing embodiments, the non-coordinating solvent is selected from the group consisting of pentanes, hexanes, heptanes, and cyclohexane. In some embodiments, including any of the foregoing embodiments, the non-coordinating solvent is heptanes. In some embodiments, including any of the foregoing embodiments, the reaction in (a) is performed at a temperature between about 50 to about 60° C., inclusive. In some embodiments, including any of the foregoing embodiments, the reaction in (a) is performed at about 55° C. In some embodiments, including any of the foregoing embodiments, the reaction in (a) is performed for at least 2 hours. In some embodiments, including any of the foregoing embodiments, (b) is performed at a temperature between about 50 to about 60° C., inclusive. In some embodiments, including any of the foregoing embodiments, the product is filtered by neutral aluminum oxide B1 filtration. In some embodiments, including any of the foregoing embodiments, the method further comprises contacting the compound:

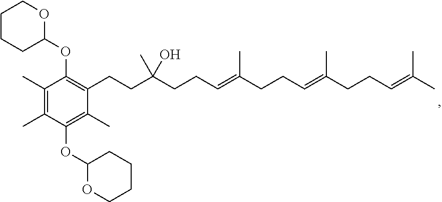 [SMILES: CC(C)=CCC/C(C)=C/CC/C(C)=C/CCC(C)(O)CCc1c(C)c(OC2CCCCO2)c(C)c(C)c1OC1CCCCO1]

or a salt thereof; and/or an isomer thereof; and/or a solvate or a hydrate thereof, with an oxidizer, wherein the oxidation product is 2-((6E,10E)-3-hydroxy-3,7,11,15-tetramethylhexadeca-6,10,14-trien-1-yl)-3,5,6-trimethylcyclohexa-2,5-diene-1,4-dione. In an alternate embodiment for producing 2-((6E,10E)-3-hydroxy-3,7,11,15-tetramethylhexadeca-6,10,14-trien-1-yl)-3,5,6-trimethylcyclohexa-2,5-diene-1,4-dione, including any of the foregoing embodiments, the method further comprises contacting the compound:

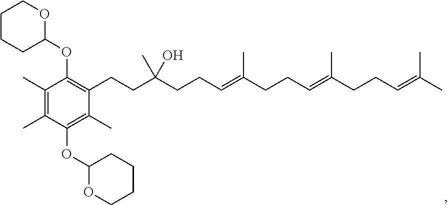 [SMILES: CC(C)=CCC/C(C)=C/CC/C(C)=C/CCC(C)(O)CCc1c(C)c(OC2CCCCO2)c(C)c(C)c1OC1CCCCO1]

or a salt thereof; and/or an isomer thereof; and/or a solvate or a hydrate thereof, with a clay, whereby the compound is converted to:

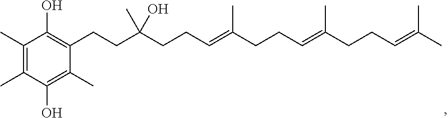 [SMILES: CC(C)=CCC/C(C)=C/CC/C(C)=C/CCC(C)(O)CCc1c(C)c(O)c(C)c(C)c1O]

followed by contacting with an oxidizer. In some embodiments, the clay is an aluminasilicate clay. In some embodiments, the clay is Montmorillonite K-10. In some embodiments, including any of the foregoing embodiments, the compound

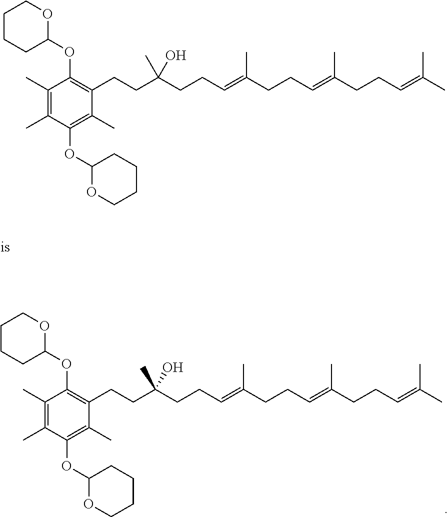 [SMILES: CC(C)=CCC/C(C)=C/CC/C(C)=C/CCC(C)(O)CCc1c(C)c(OC2CCCCO2)c(C)c(C)c1OC1CCCCO1 ; CC(C)=CCC/C(C)=C/CC/C(C)=C/CC[C@@](C)(O)CCc1c(C)c(OC2CCCCO2)c(C)c(C)c1OC1CCCCO1]

In some embodiments, including any of the foregoing embodiments, the compound

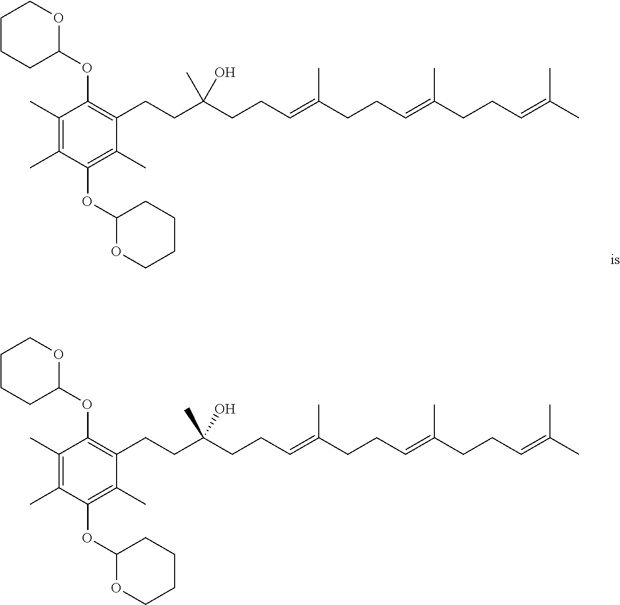 [SMILES: CC(C)=CCC/C(C)=C/CC/C(C)=C/CCC(C)(O)CCc1c(C)c(OC2CCCCO2)c(C)c(C)c1OC1CCCCO1 ; CC(C)=CCC/C(C)=C/CC/C(C)=C/CC[C@@](C)(O)CCc1c(C)c(OC2CCCCO2)c(C)c(C)c1OC1CCCCO1]

and the compound is the E-isomer at the 6 and 10-positions. In some embodiments, including any of the foregoing embodiments, the oxidizer is a Fe(III) salt. In some embodiments, including any of the foregoing embodiments, the oxidizer is iron(III) nitrate, iron(III) sulfate, iron(III) tartrate, iron(III) acetate, iron(III) citrate, iron(III) phosphate, or an iron(III) halide. In some embodiments, including any of the foregoing embodiments, the oxidizer is iron(III) chloride. In some embodiments, including any of the foregoing embodiments, the oxidation reaction is performed at a temperature between about 10 to about 30° C., inclusive. In some embodiments, including any of the foregoing embodiments, the oxidation reaction is performed at about 18° C. In some embodiments, including any of the foregoing embodiments, the oxidation reaction is performed for at least about 1 hour. In some embodiments, including any of the foregoing embodiments, the oxidation reaction is performed for about 2 hours. In some embodiments, including any of the foregoing embodiments, the oxidation reaction is performed in a mixture of iPrOAc and iPrOH. In some embodiments, including any of the foregoing embodiments, the oxidation reaction is performed in a mixture of iPrOAc and iPrOH wherein the iPrOAc:iPrOH ratio is about 1:1 (v/v). In some embodiments, including any of the foregoing embodiments, the oxidation reaction is performed in a mixture of iPrOAc and iPrOH wherein the iPrOAc:iPrOH ratio is about 1:2 (v/v). In some embodiments, including any of the foregoing embodiments, the oxidation product is filtered by SiO₂.

In some embodiments, the 2-((6E,10E)-3-hydroxy-3,7,11,15-tetramethylhexadeca-6,10,14-trien-1-yl)-3,5,6-trimethylcyclohexa-2,5-diene-1,4-dione produced by a method as described herein is formulated into a pharmaceutically acceptable composition. In some embodiments, at least about 90% of the 2-((6E,10E)-3-hydroxy-3,7,11,15-tetramethylhexadeca-6,10,14-trien-1-yl)-3,5,6-trimethylcyclohexa-2,5-diene-1,4-dione is 2-((R,6E,10E)-3-hydroxy-3,7,11,15-tetramethylhexadeca-6,10,14-trien-1-yl)-3,5,6-trimethylcyclohexa-2,5-diene-1,4-dione. In some embodiments, at least about 95% of the 2-((6E,10E)-3-hydroxy-3,7,11,15-tetramethylhexadeca-6,10,14-trien-1-yl)-3,5,6-trimethylcyclohexa-2,5-diene-1,4-dione is 2-((R,6E,10E)-3-hydroxy-3,7,11,15-tetramethylhexadeca-6,10,14-trien-1-yl)-3,5,6-trimethylcyclohexa-2,5-diene-1,4-dione. In some embodiments, at least about 98% of the 2-((6E,10E)-3-hydroxy-3,7, 11,15-tetramethylhexadeca-6,10,14-trien-1-yl)-3,5,6-trimethylcyclohexa-2,5-diene-1,4-dione is 2-((R,6E,10E)-3-hydroxy-3,7,11,15-tetramethylhexadeca-6,10,14-trien-1-yl)-3,5,6-trimethylcyclohexa-2,5-diene-1,4-dione. In some embodiments, the composition is formulated for oral or intravenous administration.

It is to be understood that the description of compounds, compositions, formulations, and methods of treatment described herein include “comprising,” “consisting of,” and “consisting essentially of” embodiments. In some embodiments, for all compositions described herein, and all methods using a composition described herein, the compositions can either comprise the listed components or steps, or can “consist essentially of” the listed components or steps. When a composition is described as “consisting essentially of” the listed components, the composition contains the components listed, and may contain other components which do not substantially affect the condition being treated, but do not contain any other components which substantially affect the condition being treated other than those components expressly listed: or, if the composition does contain extra components other than those listed which substantially affect the condition being treated, the composition does not contain a sufficient concentration or amount of the extra components to substantially affect the condition being treated. When a method is described as “consisting essentially of” the listed steps, the method contains the steps listed, and may contain other steps that do not substantially affect the condition being treated, but the method does not contain any other steps which substantially affect the condition being treated other than those steps expressly listed. As a non-limiting specific example, when a composition is described as “consisting essentially of” a component, the composition may additionally contain any amount of pharmaceutically acceptable carriers, vehicles, or diluents and other such components which do not substantially affect the condition being treated.

### BRIEF DESCRIPTION OF THE DRAWINGS

FIG. 1: NMR spectrum for Compound 3A in Example 2A.

FIG. 2: NMR spectrum for Compound 3B in Example 2B.2.

FIG. 3: NMR spectrum for Compound 4B in Example 3B.

FIG. 4: qNMR spectrum for Compound 4B in Example 3B.

FIG. 5: NMR spectrum for Compound 4B in Example 4C.

FIG. 6: qNMR spectrum for Compound 4B in Example 4C.

FIG. 7: qNMR spectrum for (R)-EE-2 in isolated fraction 2 in Example 9, Step 5.

### DETAILED DESCRIPTION

Provided herein are methods useful for the synthesis of 2-((6E,10E)-3-hydroxy-3,7,11,15-tetramethylhexadeca-6,10,14-trien-1-yl)-3,5,6-trimethylcyclohexa-2,5-diene-1,4-dione, in particular methods useful for the synthesis of 2-((R,6E,10E)-3-hydroxy-3,7,11,15-tetramethylhexadeca-6,10,14-trien-1-yl)-3,5,6-trimethylcyclohexa-2,5-diene-1,4-dione. Further provided are synthetic intermediates useful in the synthesis of 2-((6E,10E)-3-hydroxy-3,7,11,15-tetramethylhexadeca-6,10,14-trien-1-yl)-3,5,6-trimethylcyclohexa-2,5-diene-1,4-dione, in particular 2-((R,6E,10E)-3-hydroxy-3,7,11,15-tetramethylhexadeca-6,10,14-trien-1-yl)-3,5,6-trimethylcyclohexa-2,5-diene-1,4-dione, as well as methods for making said synthetic intermediates

The abbreviations used herein have their conventional meaning within the chemical and biological arts, unless otherwise specified.

Reference to “about” a value or parameter herein includes (and describes) variations that are directed to that value or parameter per se. For example, description referring to “about X” includes description of “X.” As used herein, and unless otherwise specified, the terms “about” and “approximately,” when used in connection with temperatures, doses, amounts, or weight percent of ingredients of a composition or a dosage form, mean a dose, amount, or weight percent that is recognized by those of ordinary skill in the art to provide a pharmacological effect equivalent to that obtained from the specified dose, amount, or weight percent. Specifically, the terms “about” and “approximately,” when used in this context, contemplate a temperature, dose, amount, or weight percent within 15%, within 10%, within 5%, within 4%, within 3%, within 2%, within 1%, or within 0.5% of the specified temperature, dose, amount, or weight percent

The terms “a” and “an,” as used in herein mean one or more, unless context clearly dictates otherwise

While the compounds described herein can occur and can be used as the neutral (non-salt) compound, the description is intended to embrace all salts of the compounds described herein, as well as methods of using such salts of the compounds. In some embodiments, the salts of the compounds comprise lithium salts. In some embodiments, the salts of the compounds comprise pharmaceutically acceptable salts. Pharmaceutically acceptable salts are those salts which can be administered as drugs or pharmaceuticals to humans and/or animals and which, upon administration, retain at least some of the biological activity of the free compound (non-ionic compound or non-salt compound). The desired salt of a basic compound may be prepared by methods known to those of skill in the art by treating the compound with an acid. In some embodiments, inorganic acids include, but are not limited to, hydrochloric acid, hydrobromic acid, sulfuric acid, nitric acid, and phosphoric acid. In some embodiments, organic acids include, but are not limited to, formic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, sulfonic acids, and salicylic acid. Salts of basic compounds with amino acids, such as aspartate salts and glutamate salts, can also be prepared.

The desired salt of an acidic compound can be prepared by methods known to those of skill in the art by treating the compound with a base. In some embodiments, inorganic salts of acid compounds include, but are not limited to, alkali metal and alkaline earth salts, such as sodium salts, potassium salts, magnesium salts, and calcium salts: ammonium salts: and aluminum salts. In some embodiments, organic salts of acid compounds include, but are not limited to, procaine, dibenzylamine, N-ethylpiperidine, N,N-dibenzylethylenediamine, and triethylamine salts. Salts of acidic compounds with amino acids, such as lysine salts, can also be prepared.

Included herein, when chemically relevant, are all stereoisomers of the compounds, including diastereomers and enantiomers. Also included are mixtures of possible stereoisomers in any ratio, including, but not limited to, racemic mixtures. Unless stereochemistry is explicitly indicated in a structure, the structure is intended to embrace all possible stereoisomers of the compound depicted. If stereochemistry is explicitly indicated for one portion or portions of a molecule, but not for another portion or portions of a molecule, the structure is intended to embrace all possible stereoisomers for the portion or portions where stereochemistry is not explicitly indicated.

The description of compounds herein also includes all isotopologues, in some embodiments, partially deuterated or perdeuterated analogs of all compounds herein.

The term “alkyl” is intended to embrace a saturated linear, branched, or cyclic hydrocarbon, or any combination thereof. The point of attachment of the alkyl group to the remainder of the molecule can be at any chemically possible location on the alkyl group. In some embodiments, an alkyl has from 1 to 10 carbon atoms (“C₁-C₁₀ alkyl”), or 4 to 7 carbon atoms (“C₄-C₇ alkyl”). In some embodiments, non-limiting examples of “C₄-C₇ alkyl” include n-butyl, isobutyl, sec-butyl, t-butyl, cyclobutyl, cyclopropyl-methyl, methyl-cyclopropyl, pentyl, cyclopentyl, hexyl, cyclohexyl, and heptyl.

“Cycloalkyl” in intended to embrace a monocyclic, saturated hydrocarbon radical having three to six carbon atoms. In some embodiments, cycloalkyl is cyclopropyl, cyclobutyl, cyclopentyl, or cyclohexyl.

“Racemic alpha-tocotrienol quinone” indicates the compound: 2-((R/S,6E,10E)-3-hydroxy-3,7,11,15-tetramethylhexadeca-6,10,14-trien-1-yl)-3,5,6-trimethylcyclohexa-2,5-diene-1,4-dione, where the R and S isomers are present in a ratio of about 50:50. In some embodiments, racemic alpha-tocotrienol quinone means the R:S or S:R isomers are present in a ratio of about 50:50, about 51:49, about 52:48, about 53:47, about 54:46, or about 55:45. In some embodiments, racemic alpha-tocotrienol quinone is made by the methods disclosed herein.

“Scalemic alpha-tocotrienol quinone” indicates the compound: 2-((R/S,6E,10E)-3-hydroxy-3,7,11,15-tetramethylhexadeca-6,10,14-trien-1-yl)-3,5,6-trimethylcyclohexa-2,5-diene-1,4-dione, where the R and S isomers are present. In some embodiments, scalemic alpha-tocotrienol quinone means the R:S or S:R isomers are present in a ratio of other than 50:50, for example, about 55:45, about 60:40, about 65:35, about 70:30, about 75:25, about 79:21, or 79:21. In some embodiments, scalemic alpha-tocotrienol quinone is made by the methods disclosed herein.

“Chiral alpha-tocotrienol quinone” and “enantioenriched alpha-tocotrienol quinone (or AT3Q)” is 2-((R,6E,10E)-3-hydroxy-3,7,11,15-tetramethylhexadeca-6,10,14-trien-1-yl)-3,5,6-trimethylcyclohexa-2,5-diene-1,4-dione (the naturally occurring isomer) or 2-((S,6E,10E)-3-hydroxy-3,7,11,15-tetramethylhexadeca-6,10,14-trien-1-yl)-3,5,6-trimethylcyclohexa-2,5-diene-1,4-dione. In some embodiments, chiral alpha-tocotrienol quinone, 2-((R,6E,10E)-3-hydroxy-3,7,11,15-tetramethylhexadeca-6,10,14-trien-1-yl)-3,5,6-trimethylcyclohexa-2,5-diene-1,4-dione, and 2-((S,6E,10E)-3-hydroxy-3,7,11,15-tetramethylhexadeca-6,10,14-trien-1-yl)-3,5,6-trimethylcyclohexa-2,5-diene-1,4-dione means that the R or S isomer, as applicable, is present in an amount of at least about 80% ee, at least about 85% ee, at least about 90% ee, at least about 95% ee, at least about 96% ee, at least about 97% ee, at least about 98% ee, or at least about 99% ee. In some embodiments, chiral or enantioenriched alpha-tocotrienol quinone is made by the methods disclosed herein.

“Enantioenriched” refers to a mixture of enantiomers of a compound such that the proportion of one of the enantiomers exceeds the proportion of the other (for example a 90:10 R:S mixture is enantioenriched in the R-isomer). In some embodiments, the mixture of enantiomers of a compound, in which the proportion of one of the enantiomers exceeds the proportion of the other, contains at least about 80% ee, at least about 85% ee, at least about 90% ee, at least about 95% ee, at least about 96% ee, at least about 97% ee, at least about 98% ee, or at least about 99% ee. In this application, the Examples generally provided enantioenriched compounds where the proportion of one of the enantiomers exceeded the proportion of the other in a ratio of at least about 90:10. In contrast, a racemic compound generally has approximately equal amounts of the enantiomers and is thus not enantioenriched.

### Pharmaceutical Compositions

The terms “pharmaceutical formulation” and “pharmaceutical composition” are used interchangeably herein.

The compounds described herein can be formulated as pharmaceutical compositions by formulation with additives such as pharmaceutically acceptable excipients, pharmaceutically acceptable carriers, and pharmaceutically acceptable vehicles. The terms “pharmaceutically acceptable excipients,” “pharmaceutically acceptable carriers,” and “pharmaceutically acceptable vehicles” are used interchangeably herein. Suitable pharmaceutically acceptable excipients, carriers and vehicles include processing agents and drug delivery modifiers and enhancers, such as, in some embodiments, calcium phosphate, magnesium stearate, talc, monosaccharides, disaccharides, starch, gelatin, cellulose, methyl cellulose, sodium carboxymethyl cellulose, dextrose, hydroxypropyl-β-cyclodextrin, polyvinylpyrrolidinone, low melting waxes, ion exchange resins, and the like, as well as combinations of any two or more thereof. Other suitable pharmaceutically acceptable excipients are described in “Remington's Pharmaceutical Sciences,” Mack Pub. Co., New Jersey (1991), and “Remington: The Science and Practice of Pharmacy,” Lippincott Williams & Wilkins, Philadelphia, 20th edition (2003) and 21st edition (2005), incorporated herein by reference.

A pharmaceutical composition can comprise a unit dose formulation, where the unit dose is a dose sufficient to have a therapeutic (including a suppressive) effect. The unit dose may be sufficient as a single dose to have a therapeutic (including a suppressive) effect. Alternatively, the unit dose may be a dose administered periodically in a course of treatment, prophylaxis, or suppression of a disorder.

Pharmaceutical compositions containing the compounds disclosed herein may be in any form suitable for the intended method of administration, including, in some embodiments, a solution, a suspension, or an emulsion. Liquid carriers are typically used in preparing solutions, suspensions, and emulsions. Liquid carriers contemplated for use in the practice include in some embodiments, water, saline, pharmaceutically acceptable organic solvent(s), pharmaceutically acceptable oils or fats, and the like, as well as mixtures of two or more thereof. The liquid carrier may contain other suitable pharmaceutically acceptable additives such as solubilizers, emulsifiers, nutrients, buffers, preservatives, suspending agents, thickening agents, viscosity regulators, stabilizers, and the like. Suitable organic solvents include, in some embodiments, monohydric alcohols, such as ethanol, and polyhydric alcohols, such as glycols. Suitable oils include, in some embodiments, sesame oil, soy bean oil, coconut oil, olive oil, safflower oil, cottonseed oil, and the like. For parenteral administration, the carrier can also be an oily ester such as ethyl oleate, isopropyl myristate, and the like. Compositions disclosed herein may also be in the form of microparticles, microcapsules, liposomal encapsulates, and the like, as well as combinations of any two or more thereof.

Time-release or controlled release delivery systems may be used, such as a diffusion controlled matrix system or an erodible system, as described for example in: Lee, “Diffusion-Controlled Matrix Systems”, pp. 155-198 and Ron and Langer, “Erodible Systems”, pp. 199-224, in “Treatise on Controlled Drug Delivery”, A. Kydonieus Ed., Marcel Dekker, Inc., New York 1992. The matrix may be, in some embodiments, a biodegradable material that can degrade spontaneously in situ and in vivo, in some embodiments, by hydrolysis or enzymatic cleavage, e.g., by proteases. The delivery system may be, in some embodiments, a naturally occurring or synthetic polymer or copolymer, in some embodiments, in the form of a hydrogel. Exemplary polymers with cleavable linkages include polyesters, polyorthoesters, polyanhydrides, polysaccharides, poly(phosphoesters), polyamides, polyurethanes, poly(imidocarbonates) and poly(phosphazenes).

The compounds disclosed herein may be administered enterally, orally, parenterally, sublingually, by inhalation (e.g. as mists or sprays), rectally, or topically in dosage unit formulations containing conventional nontoxic pharmaceutically acceptable carriers, adjuvants, and vehicles as desired. In some embodiments, suitable modes of administration include oral, subcutaneous, transdermal, transmucosal, iontophoretic, intravenous, intra-arterial, intramuscular, intraperitoneal, intranasal (e.g. via nasal mucosa), subdural, rectal, gastrointestinal, and the like, and directly to a specific or affected organ or tissue. For delivery to the central nervous system, spinal and epidural administration, or administration to cerebral ventricles, can be used. Topical administration may also involve the use of transdermal administration such as transdermal patches or iontophoresis devices. The term parenteral as used herein includes subcutaneous injections, intravenous, intramuscular, intra-sternal injection, or infusion techniques. The compounds are mixed with pharmaceutically acceptable carriers, adjuvants, and vehicles appropriate for the desired route of administration. Oral administration is a preferred route of administration, and formulations suitable for oral administration are preferred formulations. The compounds described for use herein can be administered in solid form, in liquid form, in aerosol form, or in the form of tablets, pills, powder mixtures, capsules, granules, injectables, creams, solutions, suppositories, enemas, colonic irrigations, emulsions, dispersions, food premixes, and in other suitable forms. The compounds can also be administered in liposome formulations. Additional methods of administration are known in the art.

In some embodiments, especially those embodiments where a formulation is used for injection or other parenteral administration including the routes listed herein, but also including embodiments used for oral, gastric, gastrointestinal, or enteric administration, the formulations and preparations used in the methods disclosed herein are sterile. Sterile pharmaceutical compositions are compounded or manufactured according to pharmaceutical-grade sterilization standards (United States Pharmacopeia Chapters 797, 1072, and 1211; California Business & Professions Code 4127.7; 16 California Code of Regulations 1751, 21 Code of Federal Regulations 211) known to those of skill in the art.

Injectable preparations, in some embodiments, sterile injectable aqueous or oleaginous suspensions, may be formulated as would be appreciated by a person of skill in the art using suitable dispersing or wetting agents and suspending agents. The sterile injectable preparation may also be a sterile injectable solution or suspension in a nontoxic parenterally acceptable diluent or solvent, in some embodiments, as a solution in propylene glycol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono or diglycerides. In addition, fatty acids such as oleic acid find use in the preparation of injectables.

Solid dosage forms for oral administration may include capsules, tablets, pills, powders, and granules. In such solid dosage forms, the active compound may be admixed with at least one inert diluent such as sucrose, lactose, or starch. Such dosage forms may also comprise additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents, such as water. Such compositions may also comprise adjuvants, such as wetting agents, emulsifying and suspending agents, cyclodextrins, and sweetening, flavoring, and perfuming agents.

The compounds disclosed herein can also be administered in the form of liposomes. As appreciated by a person of skill in the art, liposomes are generally derived from phospholipids or other lipid substances. Liposomes are formed by mono or multilamellar hydrated liquid crystals that are dispersed in an aqueous medium. Any non-toxic, physiologically acceptable and metabolizable lipid capable of forming liposomes can be used. The present compositions in liposome form can contain, in addition to a compound disclosed herein, stabilizers, preservatives, excipients, and the like. The preferred lipids are the phospholipids and phosphatidyl cholines (lecithins), both natural and synthetic. Methods to form liposomes will be appreciated by those of skill in the art. See, for example, Prescott, Ed., Methods in Cell Biology, Volume XIV, Academic Press, New York, N.W., p. 33 et seq (1976).

Also provided are articles of manufacture and kits containing materials. Also provided are kits which comprise any one or more of the compounds as described herein. In some embodiments, the kit disclosed herein comprises the container described herein.

In other aspects, the kits may be used for any of the methods described herein.

The amount of active ingredient that may be combined with the carrier materials to produce a single dosage form will vary depending upon the host to which the active ingredient is administered and the particular mode of administration. It will be understood, however, that the specific dose level for any particular patient will depend upon a variety of factors including the activity of the specific compound employed, the age, body weight, body area, body mass index (BMI), general health, sex, diet, time of administration, route of administration, rate of excretion, drug combination, and the type, progression, and severity of the particular disease undergoing therapy. The pharmaceutical unit dosage chosen is usually fabricated and administered to provide a defined final concentration of drug in the blood, tissues, organs, or other targeted region of the body. The therapeutically effective amount for a given situation can be readily determined by routine experimentation and is within the skill and judgment of the ordinary clinician.

Compounds disclosed herein may be administered in a single daily dose, or the total daily dosage may be administered in a divided dosage of two, three or four times daily. In some embodiments, dosages which can be used are a therapeutically effective amount within the dosage range of about 0.1 mg/kg to about 300 mg/kg body weight, or within about 1.0 mg/kg to about 100 mg/kg body weight, or within about 1.0 mg/kg to about 50 mg/kg body weight, or within about 1.0 mg/kg to about 30 mg/kg body weight, or within about 10 mg/kg to about 30 mg/kg body weight, or within about 10 mg/kg to about 20 mg/kg body weight, or about 15 mg/kg body weight, or within about 1.0 mg/kg to about 10 mg/kg body weight, or within about 10 mg/kg to about 100 mg/kg body weight, or within about 50 mg/kg to about 150 mg/kg body weight, or within about 100 mg/kg to about 200 mg/kg body weight, or within about 150 mg/kg to about 250 mg/kg body weight, or within about 200 mg/kg to about 300 mg/kg body weight, or within about 250 mg/kg to about 300 mg/kg body weight. In some or any embodiments, the foregoing doses are total daily doses (within a 24 hour period). In some or any embodiments, the foregoing doses are a single dose, which can be given multiple time in a day (24 hour period), in some embodiments 1, 2, or 3 times in a day (24 hour period), and in some embodiments 3 times in a day (24 hour period). In some embodiments, the total daily dose is about 100 mg, about 150 mg, about 200 mg, about 250) mg, about 300 mg, about 350) mg, about 400 mg, or about 450 mg.

While the compounds disclosed herein can be administered as the sole active pharmaceutical agent, the sole active pharmaceutical agent used to treat a particular disorder, or the sole active pharmaceutical agent in a therapeutically effective amount in a composition, they can also be used in combination with one or more other agents used in the treatment or suppression of certain disorders. Representative agents useful in combination with the compounds disclosed herein for the treatment or suppression of disorders include, but are not limited to, Coenzyme Q, vitamin E, idebenone, MitoQ, vitamins, NAC, and antioxidant compounds.

When additional active agents are used in combination with the compounds disclosed herein, the additional active agents may generally be employed in therapeutic amounts as indicated in the Physicians' Desk Reference (PDR) 53rd Edition (1999), or such therapeutically useful amounts as would be known to one of ordinary skill in the art.

The compounds disclosed herein and the other therapeutically active agents can be administered at the recommended maximum clinical dosage or at lower doses. Dosage levels of the active compounds in the compositions disclosed herein may be varied so as to obtain a desired therapeutic response depending on the route of administration, severity of the disease and the response of the patient. When administered in combination with other therapeutic agents, the therapeutic agents can be formulated as separate compositions that are given at the same time or different times, or the therapeutic agents can be given as a single composition.

### Preparation of Compounds

The methods herein advantageously do not require a plant material, such as palm oil, as a starting material. Such plant-derived materials may be expensive, and may further be subject to issues with obtaining sufficient supply required for commercial scale manufacture.

The methods described herein may further provide advantages for the synthesis of 2-((6E,10E)-3-hydroxy-3,7,11,15-tetramethylhexadeca-6,10,14-trien-1-yl)-3,5,6-trimethylcyclohexa-2,5-diene-1,4-dione, for example, for obtaining improved yields and/or purity of product, and/or by allowing for less stringent reaction conditions.

Three protecting groups were tested (see Example 2): benzyl, THP, and ethyl-TMS. As described in Example 2C, the synthetic strategies tested were not successful in producing protected 2,3,5,6-tetramethyl-benzen-1,4-diol utilizing ethyl-TMS as a protecting group. In Example 3, benzyl and THP protecting groups were tested for their ability to form the lithiated intermediates. The benzyl protecting group compound was not successful in achieving lithiation. In contrast, the THP protecting was surprisingly successful as a protecting group, achieving lithiation of up to 95% at the conditions tested. bis-THP tetramethyl hydroquinone was also easily prepared. Furthermore, the THP protecting group was readily removed by oxidation with iron(III) chloride at mild conditions (18° C.), which both removed the THP protecting group and oxidized the hydroquinone compound to the quinone product in a single step, in high yield (99%) and purity (98%) (Example 4D, one step). In an alternate method, the THP protecting group was removed by contact with Montmorillonite K-10 in order to produce the hydroquinone, which was then converted to the quinone with iron(III) chloride at mild conditions (18° C.) (Example 4D, two step). This method has the advantage of isolating the hydroquinone, which is a solid. As the quinone product is a liquid, one advantage of the two-step process is that isolation of the hydroquinone solid allows for easier purification of the hydroquinone intermediate, thus resulting, after an oxidation step, in a highly pure quinone product that does not require filtering through SiO₂ for purification.

Oxidation of the methyl-protected compound was also tested (see Example 6). Deprotection of the methyl groups was more challenging than deprotection of the THP protecting groups, requiring CAN as the oxidizing agent (and thus requiring much lower reaction temperatures). It is noted that oxidation of the methyl-protected compound with Fe(III)Cl₃ was not successful. In addition, the CAN oxidation resulted in more impurities, requiring chromatography to purify the product, thus resulting in much lower yield (59% yield in Example 6).

In the preparation of the chiral farnesyl epoxide, various lanthanum complexes were screened, but led to no major breakthrough over the use of the R-LLB complex (Example 9, Step 4). Although a few catalysts were identified of higher activity, the R-LLB complex was the best catalyst tested giving >99% conversion with 88% ee when the reaction was performed in THF at 10 mol % catalyst loading. Several alternative solvents for this reaction include toluene, MeTHF and CPME. These might be employed if it is desired to avoid the use of THE, which may be problematic on scale-up due to higher peroxide risk. In addition, several chiral phosphine oxides, instead of TMMPO, were not found to be beneficial to the reaction: surprisingly, the chirality of the selected phosphine oxide had no noticeable influence on the stereoselectively of the reaction.

The compounds disclosed herein can be prepared from readily available starting materials: non-limiting exemplary methods are described in the Examples. It will be appreciated by one of ordinary skill in the art that where typical or preferred process conditions (i.e., reaction temperatures, times, mole ratios of reactants, solvents, pressures, etc.) are given, other process conditions can also be used unless otherwise stated.

### Synthetic Reaction Parameters

The terms “solvent,” “inert organic solvent,” and “inert solvent” embrace a solvent that is inert under the conditions of the reaction being described in conjunction therewith. Solvents employed in synthesis of the compounds disclosed herein include, in some embodiments, methanol (“MeOH”), acetone, water, acetonitrile, 1,4-dioxane, dimethylformamide (“DMF”), benzene, toluene, xylene, tetrahydrofuran (“THF”), chloroform, methylene chloride (or dichloromethane, (“DCM”)), diethyl ether, pyridine and the like, as well as mixtures thereof. Unless specified to the contrary, the solvents used in the reactions disclosed herein are inert organic solvents.

The term “non-coordinating solvent” embraces solvents without available electrons to coordinate (i.e., reversibly bind) a catalyst. Examples include carbon tetrachloride, saturated hydrocarbons, fluorocarbons etc.

The term “q.s.” means adding a quantity sufficient to achieve a stated function, e.g., to bring a solution to the desired volume (i.e., 100%).

The term “eq” means an equivalent quantity of one reagent with respect to another reagent.

“Oxidation” and “oxidation step” embrace the transformation of a compound comprising a benzene-1,4-diol to a compound comprising benzoquinone. This transformation is accomplished with an oxidizer. In some embodiments, the oxidizer is selected from the group consisting of ceric ammonium nitrate, iron(III) nitrate, iron(III) sulfate, iron(III) tartrate, iron(III) acetate, iron(III) citrate, iron(III) phosphate, and/or an iron(III) halide. In some or any embodiments, the oxidizer is Fe(III)Cl₃.

Temperature in the examples below refers to bath temperature.

While the Examples illustrate some of the diverse methods available for use in assembling the compounds herein, they are not intended to define the scope of reactions or reaction sequences that are useful in preparing the compounds herein.

The disclosure will be further understood by the following non-limiting examples.

### EXAMPLES

Abbreviations

| | |
|---|---|
| 1,4- | 2,3,5,6-tetramethylbenzene-1,4-diol |
| DHTMB | |
| a/a | area/area |
| AcOH | acetic acid |
| aq | aqueous |
| Ar | argon |
| AT3 | alpha tocotrienol |
| AT3HQ | alpha tocotrienol hydroquinone |
| AT3Q | alpha tocotrienol quinone |
| BINOL | 1,1′-bi-2-naphthol |
| H⁸-binol | (R)-(+)-5,5′,6,6′,7,7′,8,8′-octahydro-1,1′-bi-2-naphthol |
| Bn | benzyl |
| bp | boiling point |
| bypr | by product |
| CAN | ceric ammonium nitrate |
| CPME | cyclopentyl methyl ether |
| DHP | 3,4-dihydro-2H-pyran |
| DIAD | diisopropyl azodicarboxylate |
| DMSO | dimethylsulfoxide |
| EtOAc | ethyl acetate |
| EtOH | ethanol |
| FA | farnesyl acetone |
| GC | gas chromatography |
| h | hour |
| HPLC | High-performance liquid chromatography |
| hyflo. | Hyflo ® Super-Cel ®, a synthetic magnesium silicate |
| IPC | in-process control |
| iPrOAc | isopropyl acetate |
| iPrOH | isopropyl alcohol |
| IT | internal temperature |
| La(O- | lanthanum(III) isopropoxide |
| ⁱPr)₃ | |
| LCMS | liquid chromatography-mass spectrometry, |
| LLB | La-Li-BINOL |
| R-LLB | 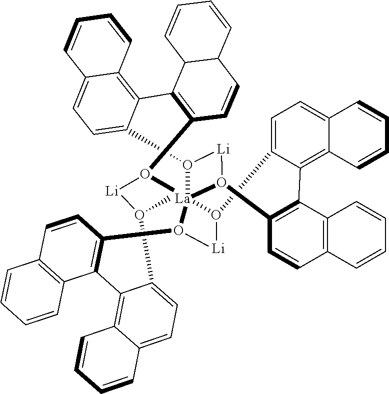 |
| MCH | methyl cyclohexane |
| MeTHF | methyl tetrahydrofuran |
| n-BuLi | n-butyl lithium |
| NH₄Cl | ammonium chloride |
| NaHCO₃ | sodium carbonate |
| Na₂SO₄ | sodium sulfate |
| nd | not determined |
| NMR | nuclear magnetic resonance |
| o/n or | overnight |
| O/N | |
| org. | organic |
| PG | protecting group |
| PTSA | para-toluenesulfonic acid |
| qNMR | quantitative nuclear magnetic resonance |
| rac | racemic |
| Rf | retention factor |
| RM | reaction mixture or raw material |
| RT | room temperature |
| rxn | reaction |
| sat. | saturated |
| SM | starting material |
| TBME | tert-butyl methyl ether |
| tBu | tert-butyl |
| t-BuLi | tert-butyl lithium |
| THF | tetrahydrofuran |
| THP | tetrahydropyran |
| TLC | thin layer chromatography |
| TMEDA | tetramethylethylenediamine |
| TMPPO | 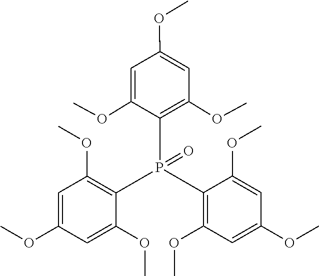 |
| TMS | trimethylsilyl |
| UPLC | ultra-performance liquid chromatography |
| Vol or | volumes |
| Vols | |
| WU | work up |

### Example 1. Synthesis of 1,4-DHTMB (2)

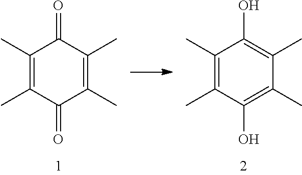

### Conditions

| | | Solvent | | | | | | Isolated |
|---|---|---|---|---|---|---|---|---|
| Ex. No. | Scale | (volumes) | Conditions (eq) | T [° C.] | t [h] | Conv. [%] | Sel. [%] | yield [%] |
| 1A | 10 g | AcOH (15) | Zn powder (4) | 25 | 17 | >99 | >99 | 96 |
| 1B | 10 g | THF (30) | H₂ (1 bar) 10 mol % Pt/C (5%- wt/wt) | 25 | 0.5 | >99 | >99 | >99 |

All solvents were degassed by bubbling Ar 15 min prior to use. All reactions were conducted under Ar. The conversion (conv. in the above table) and chemoselectivity (sel. in the above table) were determined by ¹H NMR.

### Reaction

Example 1A. 10 g of 1 were reduced as follows: 1 was stirred in AcOH at rt and in the presence of zinc powder (Strem, Mesh 325). After 17 h stirring, ⅓ of acetic acid was evaporated on a rotavapor, however the presence of the zinc hampered the evaporation. The precipitated product was dissolved adding 30 volumes THF, and the reaction mixture was filtered over hyflo. 10 volumes THF were added to wash the flask and the plug. The solvents were evaporated on a rotavapor and the obtained solid was further dried on a high vacuum to provide 9.7 g of the desired product with high purity. Product was confirmed by ¹H NMR. Example 1B. 10 g of 1 were tested under heterogeneous hydrogenation conditions using a platinum catalyst at atmospheric pressure of H₂. The reaction was directly performed in THF circumventing the use, and most importantly, removal of AcOH. Direct filtration of the catalyst followed by concentration of the reaction mixture afforded the pure product in quantitative yield and high purity. The reaction reached full conversion in 30 minutes with 10 mol % Pt/C, which indicates that lowering the catalyst loading is likely to be feasible. Product was confirmed ¹H NMR.

### Example 2. Comparison of Protecting Groups

### Example 2A. Synthesis of Bis-Bn-1,4-DHTMB (3A)

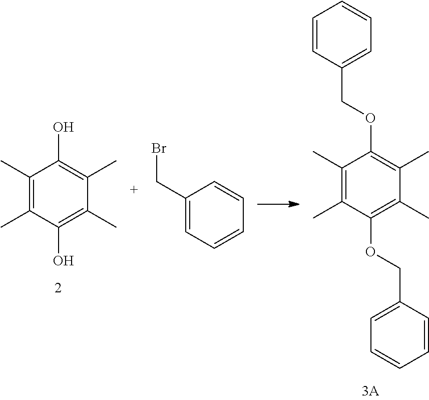

### Conditions

| | Solvent | | | | Conv. | Sel. | Isolated | |
|---|---|---|---|---|---|---|---|---|
| Scale | (volumes) | Base (eq.) | T [° C.] | t [h] | [%] | [%] | yield [%] | Comments |
| 3 g | Acetone (20) | K₂CO₃ (2.5) KI (0.1) | reflux | 17 | >99 | 75 | 53 | 3.3 g isolated HPLC purity >99% |

All solvents were degassed by bubbling Ar 15 min prior to use. All reactions were conducted under Ar. The conversion and chemoselectivity were determined by HPLC at 220 nm. Uncorrected integrals were used.

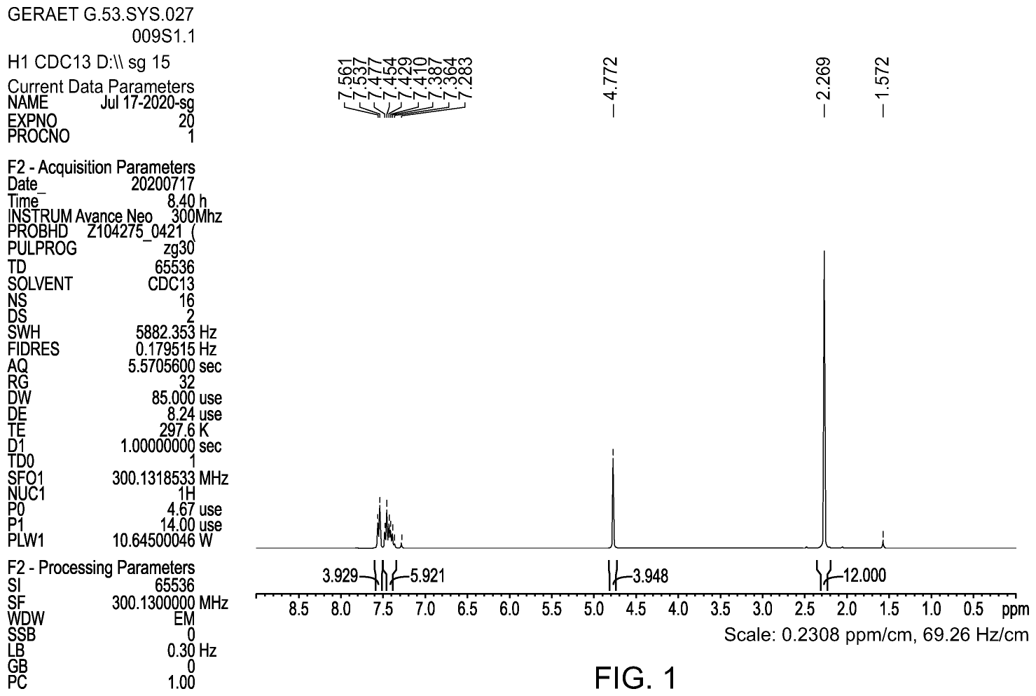

Reaction was performed using 3 g of hydroquinone 2, using acetone as solvent and K₂CO₃ as the base. 10 mol % KI was added as catalyst to promote complete conversion. No trace of starting material was detected after 17 h. The workup was hampered by the crystallization of the product in room temperature acetone. More precisely, the product started to crystallize during filtration of the reaction mixture on Hyflo (synthetic magnesium silicate), despite the filtration being done while the solution was hot. Concentration of the reaction mixture on a rotavapor to 5 volumes acetone afforded a crystalline white product in moderate yield but excellent purity (53% yield, 3.3 g with 99% purity). See FIG. 1.

### Example 2B. Synthesis of Bis-THP-1,4-DHTMB (3B)

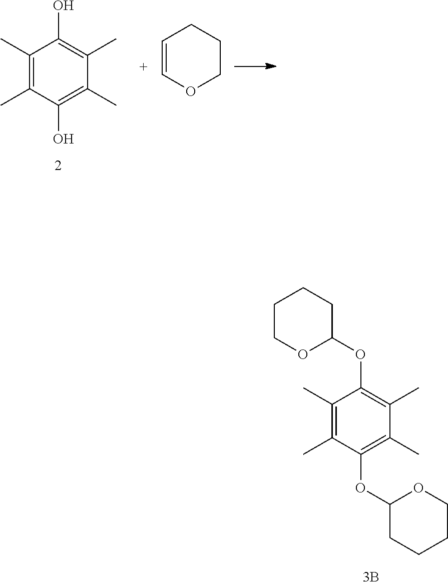

### Conditions

| Ex. | | | | | | | | Isolated | |
|---|---|---|---|---|---|---|---|---|---|
| No. | Scale | Solvent (volumes) | Catalyst (mol %) | T [° C.] | t [h] | Conv. [%] | Sel. [%] | yield[%] | Comments |
| 2B.1 | 3 g | MeTHF/Dihydropyrane (5/5) | PTSA hydrate (2) | 25 | 18 | nd | nd | 89 | 5.4 g isolated HPLC purity >99% |
| 2B.2 | 6 g | MeTHF/Dihydropyrane (5/5) | PTSA hydrate (2) | 25 | 18 | nd | nd | 86 | 10.3 g isolated HPLC purity >99% |

All solvents were degassed by bubbling Ar 15 min prior to use. All reactions were conducted under Ar. Purity of 3B was determined by HPLC at 216 nm. Uncorrected integrals were used.

Example 2B.1. The reaction was carried out in a 1:1 mixture of MeTHF/Dihydropyrane (overall 10 volumes) with a catalytic amount of para-toluenesulfonic acid hydrate. Reaction time was 18 h. Workup included washing the reaction mixture by NaHCO₃, brine and drying over Na₂SO₄. The precipitation of the product over the course of the reaction hampered the workup as large volumes of MeTHF (approx. 200 volumes) had to be employed to obtain a solution that could be neutralized with sodium bicarbonate. Upon concentration of the worked-up crude, a yellowish solid was obtained. Trituration of this solid in heptane afforded 5.4 g (89% yield) of a white solid upon filtration. The mother liquor was concentrated to obtain 2.3 g of a yellowish oil (impurities). Purity of the white solid 3B was determined to be >99% (HPLC, buffer pH 9).

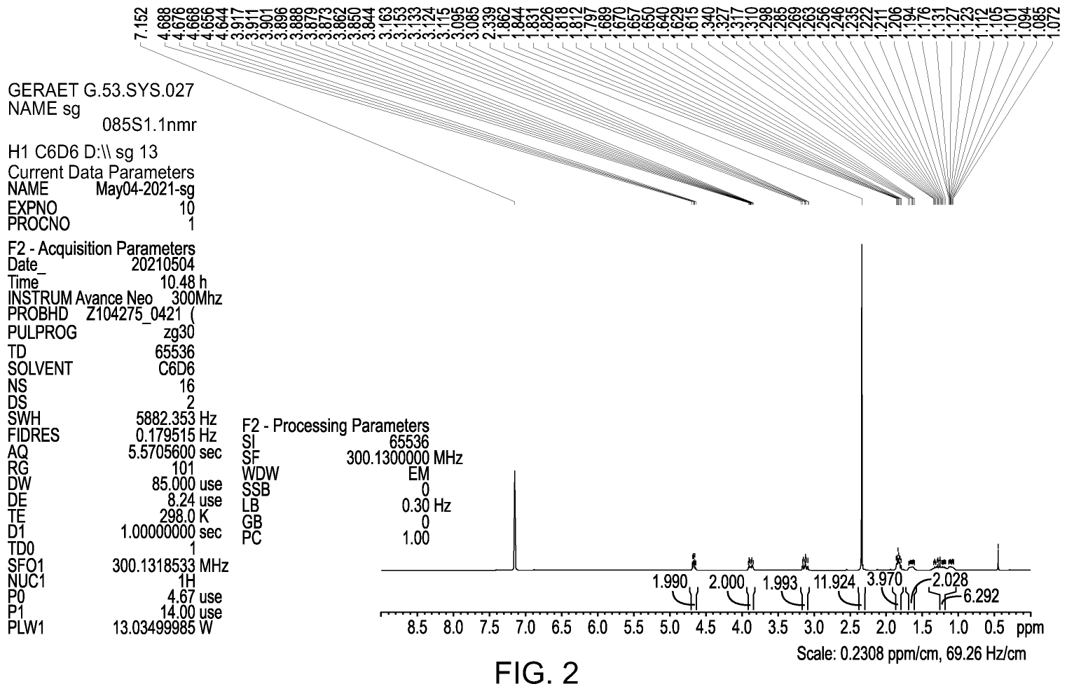

Example 2B.2. The synthesis of 3B was performed on 6 g scale. The protection reaction was found highly reliable, and provided the product with 86% yield. The purification (trituration) could also be successfully reproduced and once completed furnished 10.3 g of 3B with >99% purity (HPLC). See FIG. 2 for NMR spectrum.

### Example 2C. Synthesis of Bis-Si-1,4-DHTMB (3C)

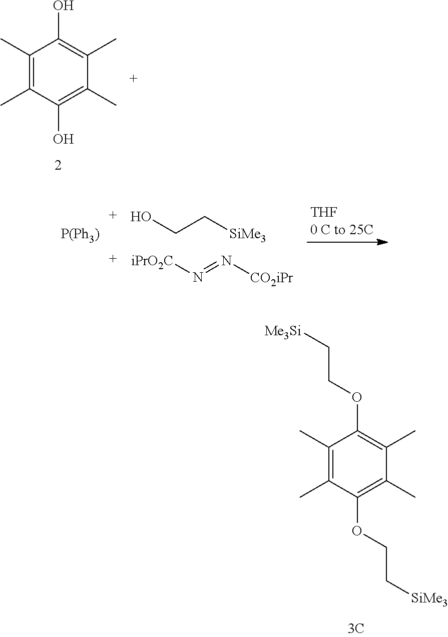

### Conditions

| Ex. | | Solvent | | | t | Conv. | Sel. | Isolated | |
|---|---|---|---|---|---|---|---|---|---|
| No. | Scale | (vols) | Conditions | T [° C.] | [h] | [%] | [%] | yield | Comments |
| 2C.1 | 250 mg | THF (20) | P(Ph₃)₃ + DIAD mixed, then | 0 to 25° C. | 18 | 100 | 0 | 0% | Full oxidation of 2 observed |
| | | | TMSEtOH added then 2 added | | | | | | |
| 2C.2 | 250 mg | THF (20) | DIAD added to a solution of 2, | 0 to 25° C. | 18 | 100 | 0 | 0% | Full oxidation of 2 observed |
| | | | P(Ph₃)₃ and TMSEtOH | | | | | | |

All solvents were degassed by bubbling Ar 15 min prior to use. All reactions were conducted under Ar. Conversion and chemoselectivity were determined by HPLC at 254/216 nm. Uncorrected integrals were used.

The protection of 2 by TMSEtOH groups under Mitsunobu conditions appeared to be not feasible. Two protocols were tested varying the addition order of starting material, but each time only oxidation of 2 to the corresponding quinone was observed by HPLC. 3C was not able to be accessed by the proposed strategy.

### Example 3. Epoxide Coupling

The coupling step was investigated using 3A (PG=Bn) and 3B (PG=THP).

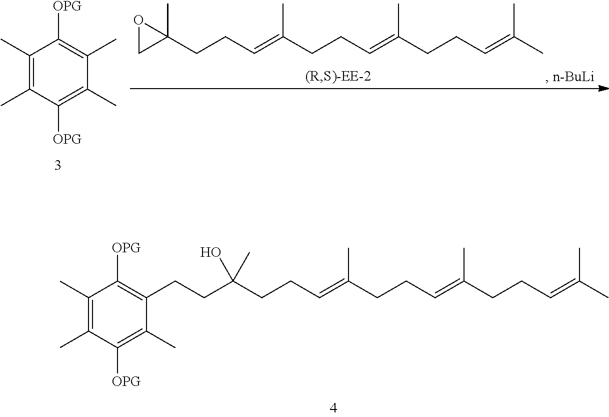

### Example 3B. Epoxide Coupling for Protected 1,4-DHTMB (3)

TABLE 1
Scouting coupling step with Compound 3B (where PG = THP).

| Exp. No. | | | Li degree | Conditions epoxide | SM 3B | Epoxide | Product | |
|---|---|---|---|---|---|---|---|---|
| (Scale) | SM | Conditions Lithiation | [%] | addition | [area %] | [area %] | [area %] | Comments |
| 012 (250 mg) | 3B | With n-BuLi 1 h at | 0 | Addition epoxide as | 0 | — | — | Poor solubility |
| | | 0° C. in MeTHF | | MeTHF solution at 0° C. | | | | of the SM in |
| | | | | stirring 4 h 0° C. | | | | MeTHF |
| 014 (250 mg) | 3B | With n-BuLi 1 h at | 49 | Addition epoxide neat at | 47 | 17 | 26 | Impurities* |
| | | 0° C. in THF | | 0° C. stirring 1 h then | | | | (area %) |
| | | then 4 h at RT | | 25° C. 4 h | | | | bypr1 (3.8) |
| | | | | | | | | bypr2 (6.1) |
| 015 (250 mg) | 3B | With n-BuLi + TMEDA | 32 | — | — | — | — | Rxn stopped |
| | | 1 h at 0° C. in THF | | | | | | after lithiation |
| | | then 4 h at RT | | | | | | |
| 016 (250 mg) | 3B | With MeLi at 45° C. | 0-10 | — | — | — | — | Rxn stopped |
| | | in THF 4 h | | | | | | after lithiation |
| 018 (250 mg) | 3B | With n-BuLi + KOtBu | 75-79 | — | — | — | — | — |
| | | 1 h at 25° C. | | | | | | |
| | | Stirring an additional | 90 (+30% | Addition epoxide as THF | 33 | 8 | 41 | Impurities* |
| | | 1 h at 25° C. | impurity | solution at 0° C. stirring | | | | (area %) |
| | | | formed) | 4 h 0° C. then RT overnight | | | | bypr1 (2) |
| | | | | | | | | bypr2 (3) |
| | | | | | | | | bypr4 (7) |
| 019 (250 mg) | 3B | With n-BuLi 17 h at | 59 | Addition epoxide neat at | 40 | 18 | 28 | Impurities* |
| | | 0° C. in THF | | 0° C. stirring 4 h . . . | | | | (area %) |
| | | | | | | | | bypr1 (6) |
| | | | | | | | | bypr2 (7) |
| | | | | . . . then stirring 2 h at | 40 | 18 | 28 | Impurities* |
| | | | | 25° C. | | | | (area %) |
| | | | | | | | | bypr1 (6) |
| | | | | | | | | bypr2 (7) |
| 022 (250 mg) | 3B | With t-BuLi 3 h | 0 | — | — | — | — | No lithiation |
| | | at −78° C. | | | | | | at low temperature |
| | | to −55° C. up to 6 h | | | | | | with t-BuLi |
| 020 (250 mg) | 3B | With n-BuLi 3 h at | 95 | Addition epoxide as THF | 22 | 9 | 59 | — |
| | | 55° C. in heptane | | solution at −20° C. | | | | |
| | | | | stirring 5 min then at RT 2 h | | | | |
| | | | | Stirring at RT 4 h | 21 | 9 | 60 | — |
| | | | | Stirring at RT overnight | 21 | 8 | 62 | Impurities* |
| | | | | (18 h) | | | | (area %) |
| | | | | | | | | bypr1 (4.1) |
| | | | | | | | | bypr2 (1.4) |
| 026 (250 mg) | 3B | With n-BuLi 3 h at | 95 | Addition epoxide as heptane | 20 | 5 | 74 | Impurities* |
| | | 55° C. in heptane | | solutionat 0° C. stirring | | | | (area %) |
| | | | | 5 min then at RT 2 h | | | | bypr1 (1.9) |
| 027 (250 mg) | 3B | With n-BuLi 3 h at | 95 | Addition epoxide as TBME | 32 | 9 | 51 | Impurities* |
| | | 55° C. in heptane | | solution at 0° C. stirring | | | | (area %) |
| | | | | 5 min then at RT 2 h | | | | bypr1 (1.9) |
| | | | | | | | | bypr2 (1.5) |
| 030 (5 g) | 3B | With n-BuLi 1 h at | 73 | — | — | — | — | |
| | | 55° C. in heptane | | | | | | |
| | | With n-BuLi 2 h at | 94 | — | — | — | — | |
| | | 55° C. in heptane | | | | | | |
| | | With n-BuLi 3 h at | 94 | Addition epoxide as heptane | 20 | 3 | 73 | Impurities* |
| | | 55° C. in heptane | | solution at 0° C. stirring 5 | | | | (area %) |
| | | | | min then at RT 2 h | | | | bypr1 (1) |
| | | | | | | | | bypr2 (1.5) |
| | | | | Stirring overnight at RT | 20 | 3 | 64 | Impurities* |
| | | | | | | | | (area %) |
| | | | | | | | | bypr1 (1) |
| | | | | | | | | bypr2 (1.5) |
| | | | | | | | | bypr3 (4.6) |

*Only impurities present >1%-a/a are discussed.

Retention time byproducts: bypr1 (1.6 min), bypr2 (6.6 min), bypr3 (4.6 min), bypr4 (13 min).

### Conditions

3B (250 mg, 0.747 mmol, 1.2 eq.), base (0.747 mmol, 1.2 eq.), farnesyl epoxide ((R,S)-EE-2) (172 mg, 0.623 mmol, 1 eq.), 12 volumes solvent. The epoxide was added as a 0.6 M solution in selected solvent.

### Calculation

The degree of lithiation was determined by ¹H NMR analysis. Conversion and chemoselectivity were determined by HPLC at 216 nm. Uncorrected integrals were used.

### Analytical Measurements

A dedicated HPLC method was developed to monitor the coupling step. The column XBridge BEH C18 2.5 μm (3.0×75 mm) was found to allow separation of 3B (peak at 3.4 min), farnesyl epoxide (5.4 min) and products of the reaction. The conversion by HPLC was measured thus far based on 3B consumption despite being used in excess, as the epoxide has a lower response factor.

As shown above in Table 1, various conditions were tested to achieve the deprotonation of 3B. The metallation was initially investigated in MeTHF, but the relative insolubility of the starting material resulted in no lithiation (exp. 012). Similar conditions were reproduced (in THF), leading to 49% lithiation degree (exp. 014). After the epoxide was added, 3 main compounds were formed. Purification by column chromatography allowed to isolate two compounds including the product rac-bis-PG-AT3HQ 4 (peak at 7.4 min) and a byproduct bypr1.

In parallel, other conditions were tested. When TMEDA was added, the lithiation was hampered (exp. 015) with 32% measured (vs. 49% for exp. 014). Using MeLi at 45° C. resulted in no lithiation (exp. 016).

When the Lochmann-Schlosser super base (mixture of nBuLi+KOtBu) was employed, 80% lithiation was achieved within 1 h even when the metallation was conducted in heptane at 0° C. Stirring the reaction mixture an additional hour enabled the lithiation to proceed further reaching 90%, however extended reaction time resulted in formation of by-products (approx. 30% based on integrated signals, ¹H NMR). Addition of the epoxide resulted in 41%-a/a product formation.

Exp. 014 was repeated allowing the lithiation to proceed over 17 h (overnight) at 0° C. No improvement was observed, the lithiation degree was nearly identical with 59% vs. 54% for the analogous exp. 014. Upon addition of the epoxide, the product was also formed in similar amounts to the previous exp. 014 (28%-a/a vs. 26% a/a for exp. 014).

Performing the reaction with t-BuLi in THF at low temperature (−78° C. to −55° C.) did not enable the lithiation of the starting material (exp. 022).

Almost complete deprotonation of 3B could be achieved when the reaction was conducted in a non-coordinating solvent such as heptane at an elevated temperature of 55° C. 95% lithiation degree was measured after 3 h reaction time. The epoxide was subsequently added as a THF solution at −20° C. and the reaction mixture was stirred 2 h at room temperature to afford the product in 59%-a/a, with 22%-a/a and 9%-a/a remaining 3B and epoxide (RS)-EE-2, respectively. Stirring the reaction overnight (18 h) did not result in increased product formation, 8%-a/a of epoxide remained unreacted. Two variations of this procedure were investigated next. After lithiation in heptane at 55° C., the epoxide was added to the reaction mixture as heptane solution in exp. 026 and as TBME solution in exp. 027. The formation of the product was favored when the epoxide was added in the non-coordinating solvent instead of THF (exp. 026, 74%-a/a product vs. exp. 020 62%-a/a product) and the reaction was cleaner. In contrast, the addition of the epoxide as TBME solution was detrimental to the coupling reaction, resulting in only 51%-a/a product (exp. 027).

Based on the promising result of exp. 026 (deprotonation+addition of epoxide in heptane), the coupling step was scaled-up engaging 5 g of 3B. The lithiation was monitored taking an IPC sample each hour. After 1 h upon addition of n-BuLi, 73% lithiation degree was observed. After 2 h, the lithiation degree attained a maximum of 94%. Stirring the reaction for an additional hour did not promote complete deprotonation of the starting material, the lithiation degree remained 94%. The epoxide (RS)-EE-2 was then added at 0° C. Similar results to the analogous small scale experiment 026 were obtained with 73%-a/a product formation and only 3%-a/a residual epoxide. However, stirring the reaction overnight at room temperature led to partial product loss (10%-a/a) with formation of impurities. This behavior was not observed on small scale. Upon workup and purification by column chromatography 71% (5.4 g) of product Rac-Bis-THP-AT3HQ 4B:

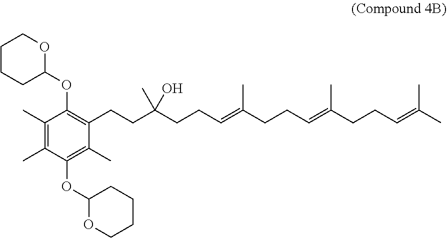 [SMILES: CC(C)=CCC/C(C)=C/CC/C(C)=C/CCC(C)(O)CCc1c(C)c(OC2CCCCO2)c(C)c(C)c1OC1CCCCO1]

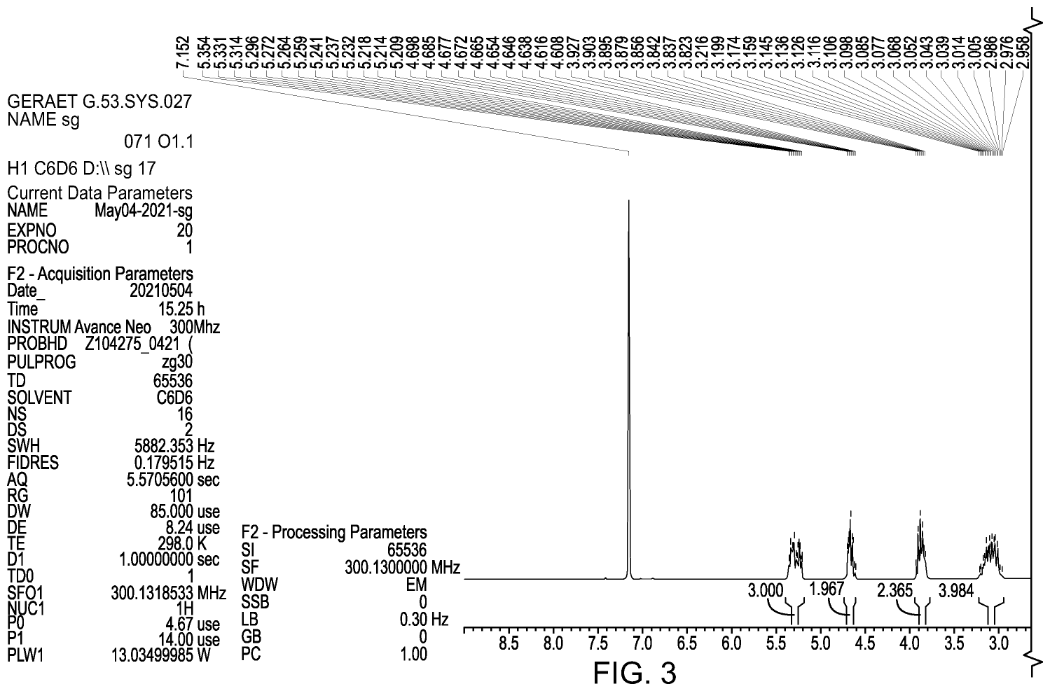

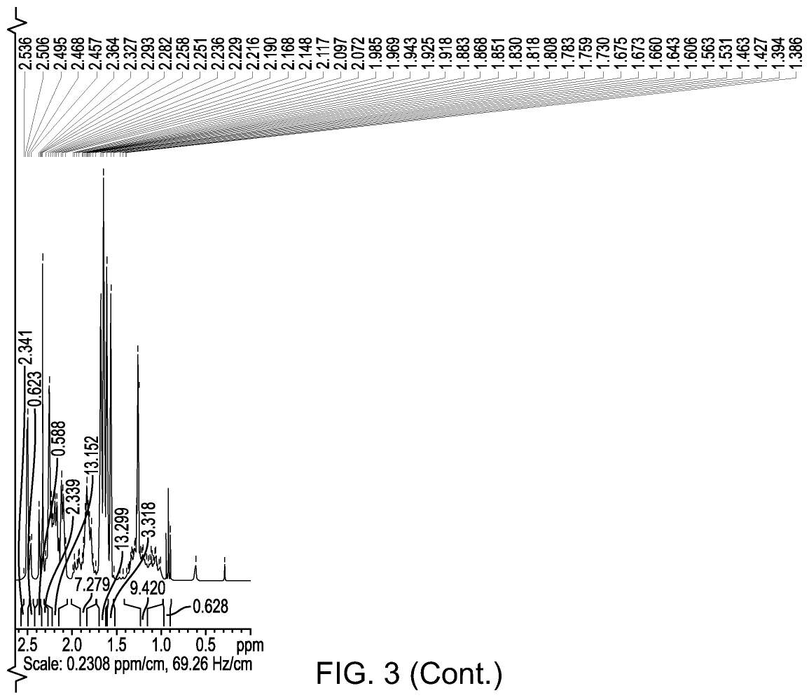

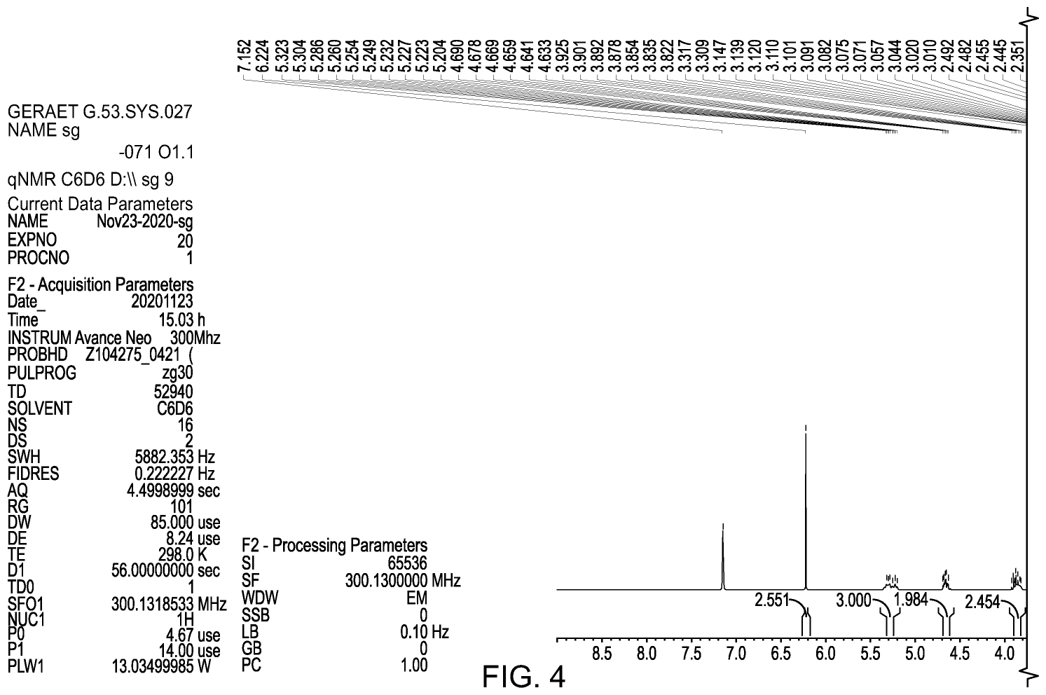

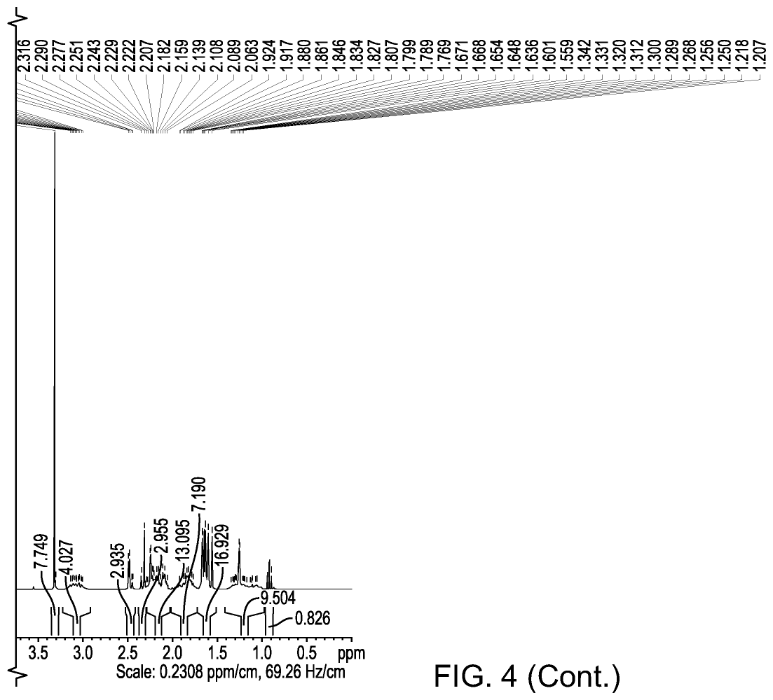

was isolated with 97% purity (HPLC). The product structure was confirmed by LCMS analysis and by NMR spectrum. See FIG. 3 for the NMR spectrum of Compound 4B. See FIG. 4 for the qNMR spectrum of Compound 4B.

### Example 3A. Epoxide Coupling for Protected 1,4-DHTMB 3A

TABLE 2
Scouting coupling step with Compound 3A (where PG = Bn).

| Exp. No. | | | Li degree | | Conv. | |
|---|---|---|---|---|---|---|
| (Scale) | SM | Conditions Lithiation | [%] | Conditions epoxide addition | [%] | Comments |
| 013 (250 mg) | 3A | With n-BuLi 1 h at | Unclear | At 0° C. then stirring 4 h | 0 | No product formed |
| | | 0° C. in MeTHF | | | | |
| 017 (250 mg) | 3A | With n-BuLi + TMEDA | Unclear | At 0° C. then stirring 4 h | 0 | No product formed |
| | | 1 h at 0° C. in THF | | | | |
| | | then 4 h at 25° C. | | | | |
| 028 (250 mg) | 3A | With n-BuLi 3 h at | 0 | — | — | No lithiation |
| | | 55° C. in heptane | | | | |
| 029 (250 mg) | 3A | With t-BuLi in heptane | 0 | — | — | No lithiation |
| | | at up to 55° C. | | | | |
| 031 (250 mg) | 3A | With n-BuLi + TMEDA | 0 | — | — | No lithiation |
| | | at up to 55° C. | | | | |

Conditions: 3A (250 mg, 0.747 mmol, 1.2 eq.), base (0.747 mmol, 1.2 eq.), farnesyl epoxide (172 mg, 0.623 mmol, 1 eq.), 12 volumes solvent. The epoxide was added as a 0.6 M solution in selected solvent.

Calculation: The degree of lithiation was determined by ¹H NMR analysis.

The lithiation of the benzyl protected hydroquinone could not be achieved using nBuLi or the combination nBuLi/TMEDA. The monitoring of the lithiation was hampered by broad signals, but addition of the farnesyl epoxide did not lead to any product formation.

The lithiation of 3A was also attempted with nBuLi in heptane at 55° C. following the procedure used to deprotonate 3B (see Experiment 020 in Example 3B above). However, no deprotonation occurred after 3 h reaction time.

The use of harsher conditions (tBuLi, exp. 029 or nBuLi/TMEDA, exp. 031: both in heptane at 55°° C.) did not enable deprotonation of 3A which remained unreactive.

### Conclusion

The epoxide coupling step was investigated under various conditions from the THP protected hydroquinone 3B and the benzyl protected hydroquinone 3A.

3B was successfully deprotonated, using n-BuLi in heptane at 55° C. High lithiation degree was achieved (94%). Subsequent addition of the farnesyl epoxide as a heptane solution instead of an ethereal solution (THF or TBME) was found to give higher conversion and chemoselectivity. Overall, the product Rac-Bis-THP-AT3HQ 4 was isolated with 71% yield (5.4 g) and 97% purity when the reaction was performed on 5 g scale.

The lithiation of 3A could not be achieved. No trace of deprotonated product was detected under the various conditions which were tested.

### Example 4. Synthesis of Rac-AT3Q (6)

### Example 4A. Synthesis of 1,4-DHTMB (2)

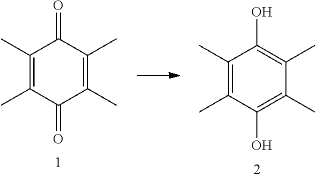

### Conditions

| | | Catalyst | | | | | | | |
|---|---|---|---|---|---|---|---|---|---|
| Exp. No. | Solvent | Loading | P H₂ | T | t | Conv. | Sel. | Isolated | |
| (Scale) | (vols) | [mol %] | [bar] | [° C.] | [h] | [%] | [%] | yield [%] | Catalyst |
| 051 (32.8 g) | THF (20) | 1 | 6 | 35 | 1 | >99 | >99 | >99 (103) | 5%-wt/wt Pt/C |

### Reaction

32.84 g (200 mmol, 1 equiv.) of 2,3,5,6-tetramethylcyclohexa-2,5-diene-1,4-dione 1 was placed in an autoclave followed by 600 mL THF. 0.328 g (2 mmol, 0.01 equiv.) of 5% Pt/C catalyst (5% Pt/C dry form) was weighted under CO₂ or argon and transferred in the autoclave with 47 mL THF.

The autoclave was closed, inertized (pressurization N₂ to 5 bar/depressurization to atmospheric pressure, 3 cycles) and flushed with H₂ (pressurization H₂ to 5 bar/depressurization to atmospheric pressure, 3 cycles). The hydrogen pressure was set to 6 bar. The valve connecting the autoclave to the reservoir was opened and the reaction mixture was stirred at 1000 rpm (note 1). The autoclave was heated to 35° C. (40° C./h ramp) until the hydrogen consumption ceased (note 2, hydrogen consumption monitored).

Heating and stirring were stopped, the autoclave depressurized and flushed three times with 5 bar nitrogen. The warm reaction mixture was filtered through a stainless steel pressure filter applying a positive pressure of N₂. The autoclave was washed with 2× 100 ml degassed THF. Each time, the washing solution was passed through the filter applying a positive pressure of N₂.

Note 1: The pressure began to drop immediately after the stirrer was started. To keep the pressure in the autoclave at a constant level, the autoclave must be connected to the hydrogen reservoir.

Note 2: The hydrogenation was complete (cease of hydrogen consumption) after the temperature of the reaction mixture reached 35° C., i.e. after ˜30 minutes after turning on stirring.

### Isolation/Workup

The product solution was concentrated to dryness on a rotary evaporator. 34.2 g of 2 (103% of the theoretical yield) was obtained as a white partially crystalline solid. The purity of the product was determined by qNMR as 97.1%. Hence, the yield corrected by purity corresponds to 100% (97.1*103/100).

The ¹H NMR spectra of the isolated product was consistent with the desired product 2.

The product was not purified and used as such in the following protection reaction (Example 4B).

### Example 4B. Synthesis of Bis-THP-1,4-DHTMB (3B)

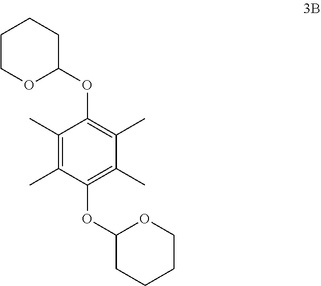 [SMILES: Cc1c(C)c(OC2CCCCO2)c(C)c(C)c1OC1CCCCO1]

| Exp. No. | | | | | Isolated | |
|---|---|---|---|---|---|---|
| (Scale) | Solvent (volumes) | Catalyst (mol %) | T [° C.] | t [h] | yield [%] | Comments |
| 058 (30 g) | MeTHF/Dihydropyrane (5/5) | PTSA hydrate (2) | 25 | 18 | 81% | 48.7 g isolated HPLC purity = 99% |

### Reaction

All solvents were degassed by bubbling Ar 15 min prior to use. All reactions were conducted under Ar.

A 1 L glass reactor equipped with a mechanical stirrer, a thermometer and gas bubbler (filled with oil) was connected to a Schlenk line and inertized by passing argon trough the reactor for 15 minutes. 2,3,5,6-Tetramethylbenzene-1,4-diol 2 (30.0 g, 180 mmol) was added, and the solid was degassed by passing argon through the reactor for an additional 15 min.

In parallel, 165 mL MeTHF and 165 mL 3,4-dihydro-2H-pyran (DHP) were mixed in a 500 mL flask and degassed by bubbling argon for 15 min through the solvent mixture. The DHP solution was transferred into the reactor and the starting material suspension was stirred (100 rpm) for 10 minutes under argon flow. Catalyst PTSA monohydrate (0.687 g, 3.61 mmol) was added in 1 portion (reaction slightly exothermic, ΔT=+5° C.). The oil bubbler was closed but a positive pressure of argon was kept maintaining the argon flow through the Schlenk line. The reaction suspension was stirred for 20 h at room temperature (20-25° C.) at 100 rpm. After 5-15 min the suspension became a yellowish solution, and a white solid precipitated again in the following 15 min.

The RM was quenched with 60 mL NaHCO₃ sat. and stirred for 30 minutes at room temperature at 200 rpm.

### Isolation Workup

The biphasic mixture was transferred into a 2 L flask and connected to a rotavapor. ⅔ of the total volume of organic solvent (approx. 250 mL) was evaporated at 45° C. under 220 to 155 mbar (approx. 45 min).

120 mL H₂O (deionized) was added to the suspension followed by 1.5 L toluene to solubilize the product (25 volumes relative to product). The biphasic mixture was transferred into a 3 L separatory funnel.

The aq. layer was discarded. The org phase was washed once more with 120 ml NaHCO₃ half sat. and the aq. phase was discarded.

The org. phase was washed with 1× 120 mL brine and the aq. phase was discarded.

The org. phase was dried over 90 g Na₂SO₄, stirred 15 min and filtered (frit P3) in a 2 L round bottom flask. The sodium sulfate was washed 2× with 50 mL toluene.

The filtrate was concentrated on a rotavapor at 45° C. to dryness (80 to 10 mbar) for 1 h to give 73 g yellowish crude product.

### Purification

900 mL heptane was added to the yellowish solid. The suspension was stirred on the rotavapor at 45° C. for 45 min, then 450 mL heptane was removed at 45° C. under 70-100 mbar over 30 minutes. Ice was added to the rotavapor bath and the suspension was stirred at 0° C. for 1 h 30 min under reduced pressure (150 mbar)

The suspension was filtered (frit P3) and washed 2× with 150 mL cold (0° C.) heptane. The white solid obtained was dried with an air flow generated by applying house vacuum for 30 min.

The white solid was transferred in a 500 mL flask for further drying on rotavapor at 45° C., 10 mbar for 30 minutes (ΔMass=300 mg). 49 g of a white solid (81% of the theoretical yield) was obtained. 15.0 g of a yellowish oily mother liquor was obtained.

The purity of the product was determined by qNMR as 99.5% and by HPLC as 98.5% (analysis at 216 nm). Hence, the yield corrected by the purity determined by qNMR corresponds to 81% (99.5*81/100).

### Example 4C. Synthesis of (6E,10E)-3,7,11,15-tetramethyl-1-(2,4,5-trimethyl-3,6-bis((tetrahydro-2H-pyran-2-yl)oxy)phenyl)hexadeca-6,10,14-trien-3-ol (4B)

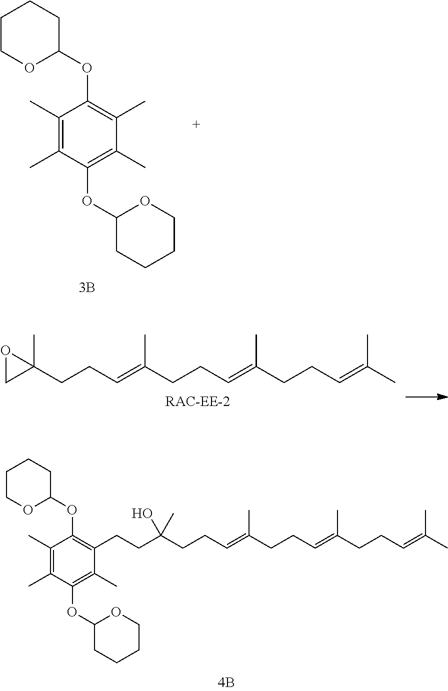 [SMILES: Cc1c(C)c(OC2CCCCO2)c(C)c(C)c1OC1CCCCO1 ; CC(C)=CCC/C(C)=C/CC/C(C)=C/CCC1(C)CO1 ; CC(C)=CCC/C(C)=C/CC/C(C)=C/CCC(C)(O)CCc1c(C)c(OC2CCCCO2)c(C)c(C)c1OC1CCCCO1]

### Reaction

A 1 L glass reactor equipped with a mechanical stirrer, a thermometer, and dropping funnel mounted with a gas bubbler (filled with oil) was connected to a Schlenk line and inertized by passing argon through the reactor for 15 min. 23.37 g 2,2′-((2,3,5,6-tetramethyl-1,4-phenylene)bis(oxy))bis(tetrahydro-2H-pyran) 3B (Example 4B) was added followed by 200 mL methyl cyclohexane and the reaction mixture was degassed by bubbling argon through the stirred (50 rpm) suspension for 30 min. n-BuLi 2.5 M in hexanes (28.0 mL, 69.9 mmol, 1.4 equiv.) was added to the white suspension in one portion (in less than 5 minutes, addition slightly exothermic, ΔT=2° C.) and the reaction was heated to 55° C. IT (oil bath was pre-heated at 58-60° C.) in 15-20 minutes. The RM was stirred at 55° C. at 100 rpm for up to 2 h 40 min. The lithiation was monitored by ¹H NMR.

IPC 1—1 h 20 min after the reaction mixture reached 55° C. Sample preparation: 200 μL of the RM quenched in 750 μL CD₃OD placed in an HPLC vial. Suspension transferred in a 10 mL flask, concentrated in rotavapor and diluted in 750 μL C₆D₆. approx. 87% lithiation degree determined.

IPC 2—2 h 40 min after the reaction mixture reached 55° C. Sample preparation: 200 μL of the RM quenched in 750 μL CD₃OD placed in an HPLC vial. Suspension transferred in a 10 mL flask, concentrated in rotavapor and diluted in 750 μL C₆D₆. >95% lithiation degree determined.

In parallel, a solution of 2-methyl-2-((3E,7E)-4,8,12-trimethyltrideca-3,7,11-trien-1-yl)oxirane RAC-EE-2 (15 g, 49.9 mmol, 1 equiv.) in 40 mL methyl cyclohexane was prepared in a 100 mL flask and degassed by bubbling argon for 15 minutes.

Once the lithiation of 3B was confirmed to be complete the epoxide solution was placed in the dropping funnel and added in one portion (<5 min) to the bright orange Li-3B solution (reaction highly exothermic, ΔT=+15° C.). The reaction mixture was stirred 40 min at 55° C. The epoxide consumption was monitored by HPLC.

IPC 3—40 min after addition of the epoxide. Sample preparation: 200 μL of the reaction mixture quenched in 750 μL sat. NH₄Cl. 10 μl of the org. phase diluted in 1 mL MeCN for HPLC analysis with method 2 at 216 nm. Full conversion epoxide observed.

The reaction mixture was cooled down to 10° C. with an ice bath and 100 mL of a half saturated solution of NH₄Cl was added in one portion (addition slightly exothermic, ΔT=+5° C.). The ice bath was removed and the biphasic reaction mixture was vigorously stirred at 150 rpm for 30 min.

### Isolation Workup

The biphasic mixture was transferred into a 1 L separatory funnel. The aqueous and organic layers were separated. (HPLC of the obtained aq. phase after separation showed a trace of product).

The reactor was washed with 40 mL MCH. The washing solution was used to extract the aqueous phase (HPLC of the obtained aq. phase after separation showed a trace of product). The organic phases were combined.

The reactor was washed with 50 mL MCH. The washing solution was used to extract the aqueous phase (HPLC of the obtained aq. phase after separation showed no trace of product). The organic phases were combined.

IPC 4—Analysis of the combined organic phases. Sample preparation: 10 μL of the org. phase diluted in 1 mL MeCN for HPLC analysis with method 2 at 216 nm. Chromatogram identical to IPC 3.

The combined organic phase (350 mL) were stored at RT overnight.

### Purification

The purification of the product was achieved by neutral aluminum oxide B1 filtration. To facilitate the purification, the combined org. phase (350 mL) were split in two equal portions and two filtrations (identical setup) were conducted.

The plug was prepared adding 1.4 kg neutral alox B1 in a column (Alox plug, ø=15 cm, h=6.5 cm), then packed using MCH and layered with 1 cm of sea sand. 175 mL of the org. phase obtained after workup was directly introduced on the plug, washed with 2×250 mL MCH. Then a mixture of 5% EtOAc in MCH was used to elute the starting material. Five 900 mL fractions were collected and checked by TLC (alox neutral, eluted with 5% EtOAc in MCH). Upon confirmation no starting material was eluting, the eluent was swapped by pure ethyl acetate and three 900 mL fractions were collected and checked by TLC (alox neutral, eluted with 5% EtOAc in MCH). All fractions containing the product were concentrated on a rotavapor at 45° C., up to 10 mbar.

Overall 26.3 g of a yellowish oil (86% of the theoretical yield) was obtained combining the material obtained from the two plug filtrations.

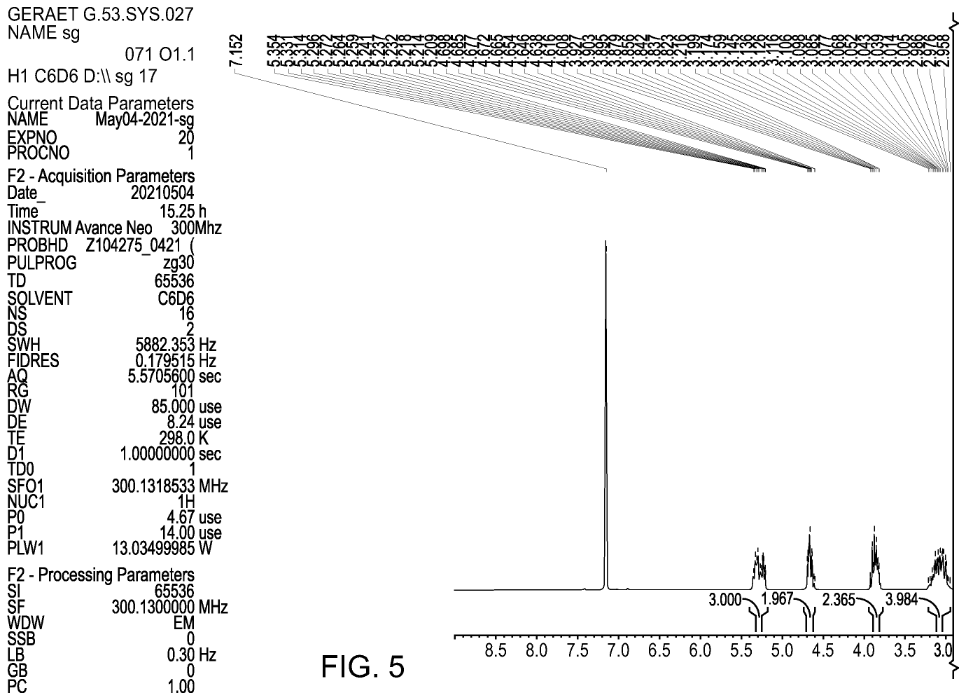

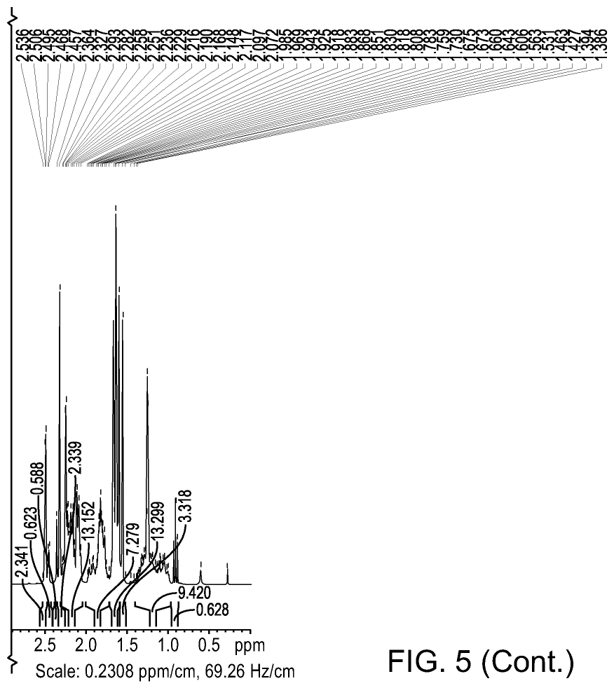

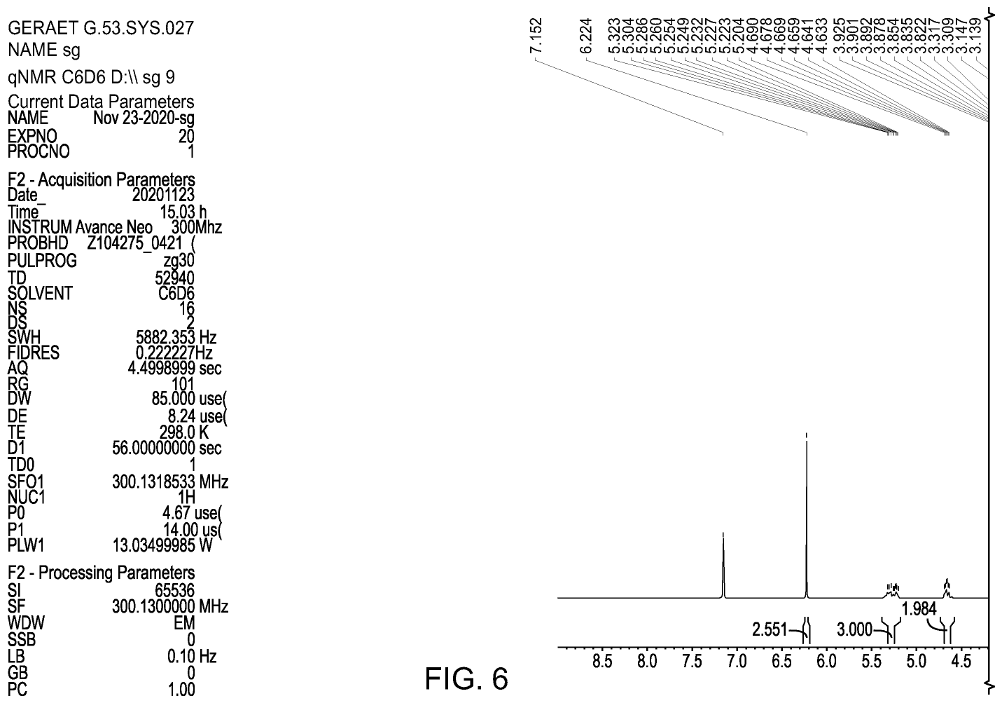

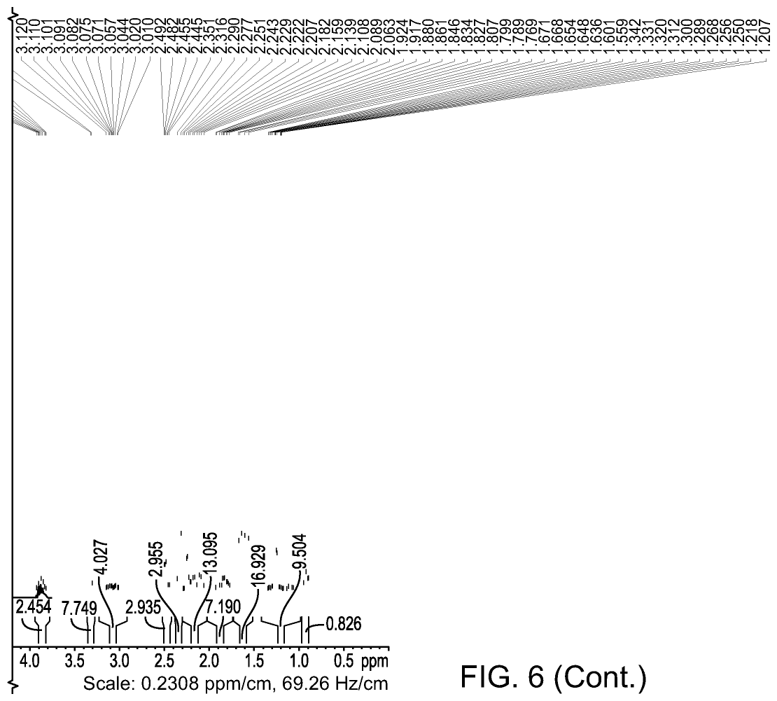

The identity of the product was confirmed by LCMS and NMR (FIG. 5). The purity of the product was determined by qNMR (FIG. 6) as 95.1%, corrected to 99.6% (as 4.5% EtOAc was present and could be quantified by qNMR) and by HPLC as >99% (analysis at 216 nm). Hence, the yield corrected by the purity determined by qNMR corresponds to 82% (95*86/100).

### Example 4D. Synthesis of 2-((6E,10E)-3-hydroxy-3,7,11,15-tetramethylhexadeca-6,10,14-trien-1-yl)-3,5,6-trimethylcyclohexa-2,5-diene-1,4-dione (6) Single Step Method

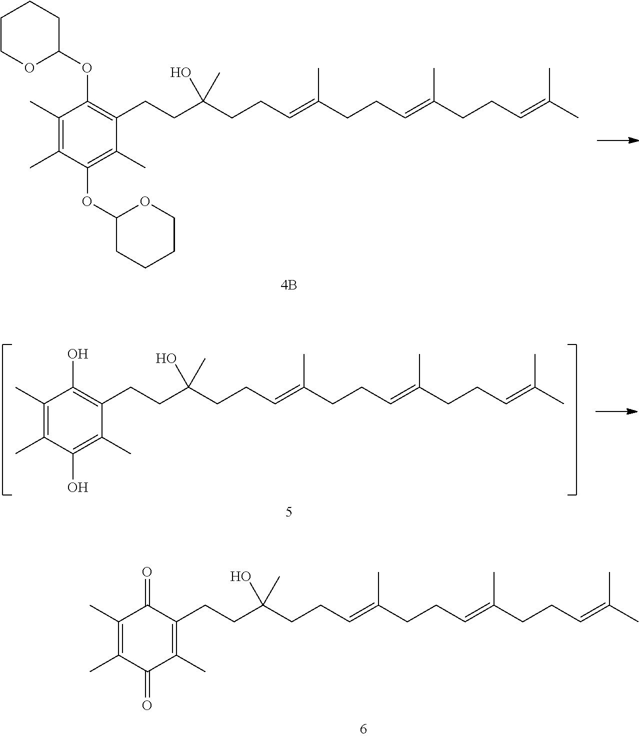

### Reaction

(6E,10E)-3,7,11,15-tetramethyl-1-(2,4,5-trimethyl-3,6-bis((tetrahydro-2H-pyran-2-yl)oxy)phenyl)hexadeca-6,10,14-trien-3-ol 4B (Example 4C) (20 g, 31.3 mmol) was placed in a 500 mL amber flask and 40 mL iPrOAc followed by 44 mL iPrOH were added and the reaction was stirred at 18° C. to 22° C. (ambient temperature) in a water bath to control the temperature.

FeCl₃.6H₂O (46.5 g, 172 mmol, 5.5 eq.) was dissolved in deionized water (52.4 ml, 2908 mmol, 93 equiv.) by stirring the iron mixture for 10 min at RT. Once a solution was obtained, the iron solution was added (ΔT=+2° C.) to the reaction mixture which was vigorously stirred at 18° C. for exactly 1 h 30 min.

No IPC was taken. The reaction was worked up after 1 h 30 min.

### Workup

The biphasic mixture was transferred into a 250 mL amber separatory funnel. The aq. and org. layers were separated. The aq. layer was extracted 3× with 20 mL iPrOAc. The org. phases were combined.

The combined org. phases were washed 1× with 20 mL deio. water and 1× with 10 mL deionized water. The combined aq. phases were back extracted 2× with 2500 μL iPrOAc.

The combined org. phases were washed 2× with 20 mL 8% wt/wt citric acid monohydrate aq. solution. The combined the combined citric acid phases were back extracted 1× with 2500 μL iPrOAc.

The combined org. phases were washed 2× with 20 mL 10% wt/wt NaCl solution. Volume org. phase=approx. 250 ml. The organic phase was diluted by 70 mL heptane.

### Isolation

A plug of SiO₂ 45 g (3 wt/expected mass of product if yield is 100%) was prepared using pure heptane. The crude product solution was added on the plug, and the product was then eluted with approx. 500 ml 10% iPrOAc/heptane until the yellow layer completely eluted.

The solution was concentrated on a rotavapor at 45° C. and dried at 10 mbar for 15 min. Then, the product was co-evaporated twice with 50 mL 1:1 iPrOH/iPrOAc, and further dried at 10 mbar for 1 h.

Overall 13.68 g of a yellowish oil (99% of the theoretical yield) was obtained.

The purity of the product 6 was determined by qNMR as 97.6% as well as HPLC using two different methods as 98.9% and 99% respectively (analysis at 254 nm). Hence, the yield corrected by purity (qNMR, 97.6*99/100) corresponds to 98%.

### Two Step Method

In an alternate method, the hydroquinone (5) was isolated prior to oxidation to the quinone (6). Compound 4B was converted to the hydroquinone (5) as follows:

| | Solvent | | T | t | Conv. | Product | Crude |
|---|---|---|---|---|---|---|---|
| -(Scale) | (volumes) | Conditions | [° C.] | [h] | [%] | [area %] | yield [%] |
| 034 (500 mg) | MeOH (20) | Montmorillonite K-10 (100%- wt/wt/SM) | 25 | 2 | >99 | 95 | 120 |

All solvents were degassed by bubbling Ar 15 min prior to use. All reactions were conducted under Ar.

Calculation: The conversion and chemoselectivity were determined by HPLC at 216 nm. Uncorrected integrals were used. All HPLC samples were prepared in degassed MeCN.

The solvent (methanol) used in all experiments was degassed to monitor the deprotection avoiding in situ oxidation of the unprotected product. The reaction was conducted under argon and all HPLC samples were prepared in degassed MeCN.

The deprotection was found to be promoted by Montmorillonite K-10. Complete conversion of the starting material was reached in only 2 h. Selective formation of the hydroquinone Rac-AT3HQ 5 was observed (95%-a/a). The reaction mixture was filtered to remove the clay, then concentrated on a rotavapor and immediately used in the next oxidation step.

Hydroquinone 5 was converted to quinone 6 as follows:

| | Solvent | | T | t | Conv. | Product | Isolated | |
|---|---|---|---|---|---|---|---|---|
| (Scale) | (volumes) | Conditions (eq.) | [° C.] | [h] | [%] | [area %] | yield [%] | Comments |
| 046 (500 mg) | iPrOAc (0.7) | FeCl₃.6H₂O aq. sol (5.5) | 18 | 2 | >99 | 91 | 78 | Isolated Product purity 98.8% (HPLC) |
| | iPrOH (1.4) | | | | | | | |

Calculation: The conversion and chemoselectivity were determined by HPLC at 254/216 nm. Uncorrected integrals were used. All HPLC samples were prepared in degassed MeCN.

A work up was performed as follows: After reaction completion, the aqueous phase was separated and the organic phase was dried over a mixture of sodium bicarbonate and sodium sulfate, then filtered and concentrated. The product was filtered through SiO₂ and isolated.

### Example 5. Synthesis of Enantioenriched (R)-AT3Q (6)

The synthesis of enantioenriched (R)-AT3Q (6) was conducted, using the enantioenriched epoxide produced as described in Example 9 (91.5% ee), following identical conditions as described in Example 4.

### Example 5A. Synthesis of (R)-bis-THP-AT3HQ (4B)

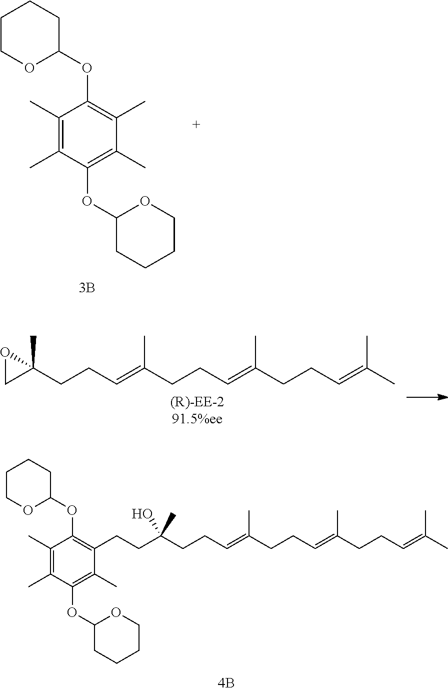 [SMILES: Cc1c(C)c(OC2CCCCO2)c(C)c(C)c1OC1CCCCO1 ; CC(C)=CCC/C(C)=C/CC/C(C)=C/CC[C@]1(C)CO1 ; CC(C)=CCC/C(C)=C/CC/C(C)=C/CC[C@@](C)(O)CCc1c(C)c(OC2CCCCO2)c(C)c(C)c1OC1CCCCO1]

### Conditions

| Exp. No. | | | Conditions epoxide | Epoxide | Product | |
|---|---|---|---|---|---|---|
| (Scale) | Conditions | Li degree/t | addition | [area %]^{a} | [area %]^{a} | Comment |
| 077 (12.2 g | 1.4 eq. 3B | >95%/2 h 40 min | Addition epoxide in 3 vol. | 0 | 80 | Chemical purity 97.6% |
| 3B/8 g epoxide | 1.4 eq. nBuLi | | MCH at 55° C., 30 min | | | Impurities (area %) |
| | 55° C., 3 h | | | | | bypr 1.3 min (0.6) |
| | Me-cyclohexane | | | | | bypr 6.4 min (1.3) |
| | (10 vol.) | | | | | 13.6 g isolated |

^{a}Values given at 216 nm.

Calculation: The lithiation degree was determined by ¹H NMR analysis.

The synthesis of enantioenriched 4B was conducted engaging 12.2 g of 3B and 8 g of the enantioenriched epoxide (R)-EE-2. 13.6 g of enantioenriched 4B was isolated (86% yield) and >99% purity (HPLC). ¹H NMR analysis showed the presence of 5.8%-wt/wt EtOAc, which gave a corrected yield of 81%.

### Example 5B. Synthesis of 2-((R,6E,10E)-3-hydroxy-3,7,11,15-tetramethylhexadeca-6,10,14-trien-1-yl)-3,5,6-trimethylcyclohexa-2,5-diene-1,4-dione (6)

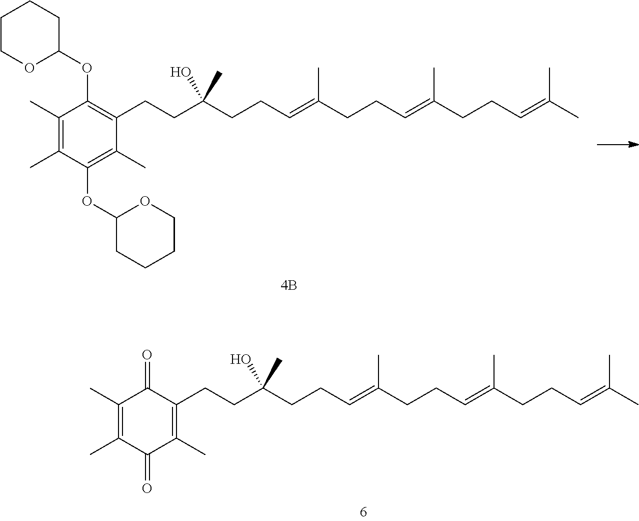

### Conditions

| Exp. No. | Solvent | | | T | t | |
|---|---|---|---|---|---|---|
| (Scale) | (volumes) | Conditions (eq.) | | [° C.] | [h] | Isolated yield [%] |
| 078 (12.8 g) | iPrOAc (2.2) | FeCl₃.6H₂O aq. sol (5.5) | 18 | 2 | 97% | 8.9 g isolated With 91.5% ee |
| | iPrOH (2.2) | | | | | |

All reactions and workups were conducted in amber glassware.

Calculation: The purity was determined by HPLC at 254 nm. Uncorrected integrals were used. All HPLC samples were prepared in degassed MeCN in amber vials. The enantiomeric excess was determined by chiral HPLC method at 265 nm.

12.8 g of starting material was engaged and 8.9 g product was isolated corresponding to 97% yield. The purity was high with 99% determined by HPLC and 99.4% purity determined by qNMR. Analysis of the product with the chiral HPLC method indicated 91.5% ee. This enantiomeric excess is in line with the optical purity of the enantioenriched epoxide used in the previous coupling step. Hence, no erosion of optical purity occurred during the coupling step or final deprotection/oxidation reaction.

A sample of AT3Q 6 obtained from Example 4D as well as the above enantioenriched (R)-AT3Q (6) was analyzed by UPLC. The sample from Example 4 showed 93.6% purity, which was almost identical to the enantioenriched sample with 93.1% purity.

Previous analyses of AT3Q 6 obtained from Example 4D indicated 97.6% purity by qNMR and 98.9% purity by HPLC (method 2). The purity of Example 5B was 99.4% by qNMR and 99% by HPLC (method 2). The difference can be explained by the higher sensitivity of the UPLC method. One impurity was detected when HPLC method 2 was used while 12 impurities were observed using the UPLC, albeit generally in low amounts, ranging from 0.1% to 1.8%-a/a.

### Example 6. Oxidation of (R,6E,10E)-1-(2,5-dimethoxy-3,4,6-trimethylphenyl)-3,7,11,15-tetramethylhexadeca-6,10,14-trien-3-ol

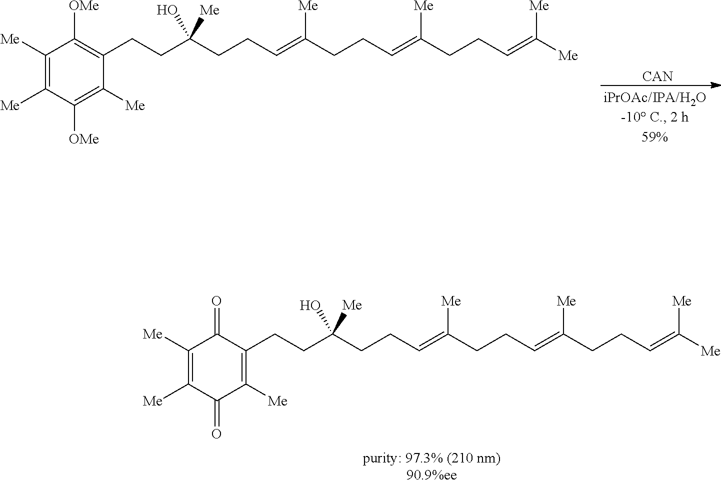 [SMILES: COc1c(C)c(C)c(OC)c(CC[C@](C)(O)CC/C=C(\C)CC/C=C(\C)CCC=C(C)C)c1C ; "CAN" ; CC(C)=CCC/C(C)=C/CC/C(C)=C/CC[C@@](C)(O)CCC1=C(C)C(=O)C(C)=C(C)C1=O]

To a 100 mL flask was charged (R,6E,10E)-1-(2,5-dimethoxy-3,4,6-trimethylphenyl)-3,7,11,15-tetramethylhexadeca-6,10,14-trien-3-ol (2.00 g, 4.25 mmol, 1.0 eq.), i-PrOAc (14.0 g) and IPA (6.00 g). After cooling the clear solution to −10° C., ceric ammonium nitrate (11.65 g, 21.24 mmol, 5.0 eq.) in H₂O (20.0 g) was added slowly. After stirring the resulting red solution at −10° C. for 2 h, the mixture was warmed to room temperature and the aqueous layer was separated. The resulting organic layer was washed with H₂O (20.0 g) and 10% Na₂CO₃ aq. (20.0 g) and dried over Na₂SO₄. After concentration in vacuo, the resulting residue was purified by silica gel chromatography (EtOAc/n-hexane=20:80) to give product (1.10 g, 59%, 90.9% ee) as orange oil (purity: 97.3% (determined by HPLC analysis at 210 nm)).

### Example 7. Synthesis of RAC-EE-2

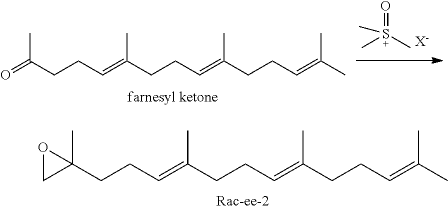

| -Exp. No. | Ylide salt | Conditions Ylide | Conditions Farnesyl | SM 3B | Epoxide | |
|---|---|---|---|---|---|---|
| (Scale) | precursor (X) | formation | acetone addition | [area %] | [area %] | Comments |
| 076 (0.88 g) | chloro | 1.1 eq. S⁺ salt | Addition ylide sol. to | 22.2 (GC) | 76.9 (GC) | |
| | | 1.05 eq. KOtBu | FA sol. (1 vol.) at 25° C., | 32.2 (HPLC) | 66.2 (HPLC) | |
| | | 3.2 vol. DMSO | stirring 1 h | | | |
| | | (/FA) 25° C., 1 h | | | | |
| | | | stirring 20 h | 3.8 (GC) | 94.9 (GC) | 1.3%-a/a Impurities (GC) |
| | | | | 6.4 (HPLC) | 89.9 (HPLC) | |
| 077 (0.88 g) | chloro | 1.1 eq. S⁺ salt | Addition FA sol. (1 vol.) | 17.8 (GC) | 81.3 (GC) | |
| | | 1.05 eq. KOtBu | to ylide sol. at 25° C., | 24.6 (HPLC) | 74.2 (HPLC) | |
| | | 3.2 vol. DMSO | stirring 1 h | | | |
| | | (/FA) 25° C., 1 h | | | | |
| | | | stirring 20 h | 1.9 (GC) | 96.0 (GC) | 2.1%-a/a Impurities (GC) |
| | | | | 2.9 (HPLC) | 92.8 (HPLC) | |
| 078 (0.88 g) | chloro | 1.4 eq. S⁺ salt | Addition FA neat to | 36.0 (GC) | 61.0 (GC) | |
| | | 1.35 eq. KOtBu | ylide sol. at 25° C., | 43.0 (HPLC) | 54.2 (HPLC) | |
| | | 5 vol. THF (/FA) | stirring 1 h | | | |
| | | 65° C., 5 h | | | | |
| | | | stirring 20 h | 4.4 (GC) | 88.7 (GC) | 11.3%-a/a Impurities (GC) |
| | | | | 0.0 (HPLC) | 88.7 (HPLC) | |
| 079 (0.88 g) | chloro | 1.4 eq. S⁺ salt | Addition FA neat to | 43.3 (GC) | 53.6 (GC) | |
| | | 1.35 eq. KOtBu | ylide sol. at 25° C., | 49.2 (HPLC) | 47.9 (HPLC) | |
| | | 5 vol. MeTHF | stirring 1 h | | | |
| | | (/FA) 65° C., 5 h | | | | |
| | | | stirring 20 h | 5.6 (GC) | 88.7 (GC) | 11.3%-a/a Impurities (GC) |
| | | | | 0.0 (HPLC) | 83.9 (HPLC) | |
| 080 (0.88 g) | iodo | 1.2 eq. S⁺ salt | Addition FA sol. (1 vol.) | 13.3 (GC) | 85.6 (GC) | |
| | | 1.15 eq. KOtBu | to ylide sol. at 25° C., | 19.3 (HPLC) | 80.1 (HPLC) | |
| | | 3.2 vol. DMSO | stirring 1 h | | | |
| | | (/FA) 25° C., 1 h | | | | |
| | | | stirring 20 h | 0.6 (GC) | 96.4 (GC) | 3.6%-a/a Impurities (GC) |
| | | | | 0.0 (HPLC) | 95.7 (HPLC) | |
| 081 (0.88 g) | iodo | 1.2 eq. S⁺ salt | Addition FA neat to | 11.3 (GC) | 87.5 (GC) | |
| | | 1.15 eq. KOtBu | ylide sol. at 25° C., | 16.8 (HPLC) | 82.5 (HPLC) | |
| | | 3.2 vol. DMSO | stirring 1 h | | | |
| | | (/FA) 25° C., 1 h | | | | |
| | | | stirring 20 h | 0.6 (GC) | 95.8 (GC) | 4.2%-a/a Impurities (GC) |
| | | | | 0.0 (HPLC) | 96.8 (HPLC) | |
| 082 (0.88 g) | iodo | 1.2 eq. S⁺ salt | Addition FA neat to | 12.6 (GC) | 86.1 (GC) | |
| | | 1.15 eq. KOtBu | ylide sol. at 0° C., | 19.2 (HPLC) | 78.9 (HPLC) | |
| | | 3.2 vol. DMSO | stirring 1 h at 25° C. | | | |
| | | (/FA) 25° C., 1 h | | | | |
| | | | stirring 20 h | 0.6 (GC) | 96.2 (GC) | 3.8%-a/a Impurities (GC) |
| | | | | 0.0 (HPLC) | 95.9 (HPLC) | |
| 083 (0.88 g) | iodo | 1.2 eq. S⁺ salt | Addition FA neat to | 15.8 (GC) | 83.1 (GC) | |
| | | 1.15 eq. KOtBu | ylide sol. at 25° C., | 21.6 (HPLC) | 78.3 (HPLC) | |
| | | 1.6 vol. DMSO | stirring 1 h | | | |
| | | (/FA) 1.6 vol. MeTHF | | | | |
| | | (/FA) 25° C., 1 h | | | | |
| | | | stirring 20 h | 0.5 (GC) | 97.5 (GC) | 2.5%-a/a Impurities (GC) |
| | | | | 0.0 (HPLC) | 98.5 (HPLC) | |
| 084 (25 g) | iodo | 1.2 eq. S⁺ salt | Addition FA neat to | 14.8 (GC) | 82.4 (GC) | |
| | | 1.15 eq. KOtBu | ylide sol. at 25° C., | | | |
| | | 3.2 vol. DMSO | stirring 1 h | | | |
| | | (/FA) 25° C., 1 h | | | | |
| | | | stirring 20 h | 0.5 (GC) | 97.5 (GC) | 28 g crude (decantation) |
| | | | | | | 22.5 g product (after WU) |
| | | | | | | 17 g product (after distil.) |
| 087 (25 g) | iodo | 1.2 eq. S⁺ salt | Addition FA neat to | — | — | |
| | | 1.15 eq. KOtBu | ylide sol. at 25° C., | | | |
| | | 3.2 vol. DMSO | stirring 1 h | | | |
| | | (/FA) 25° C., 1 h | | | | |
| | | | stirring 20 h | 0.5 (GC) | 96.7 (GC) | 22.5 g product (after WU) |

Conditions: Farnesyl acetone (0.88 g, 1 mL, 3.35 mmol, 1 eq.), base (3.86 mmol, 1.15 eq., unless indicated otherwise), trimethylsulfoxonium halide (4.02 mmol, 1.2 eq. unless indicated otherwise), [C₀]=as indicated.

Calculation: All reactions were monitored by HPLC at 216 nm and GC. Uncorrected integrals were used.

### General Comments

The same sample showed 99.5% purity when analyzed with HPLC. The results were not corrected by the initial purity of the starting material. All reactions were monitored by GC and HPLC.

### Comments on Table

Experiments 076 and 077. The base (1.05 eq.) was added to a DMSO solution (3.2 vol./Farnesyl acetone) of the trimethylsulfoxonium chloride (1.1 eq.). The suspension was stirred for 1 h to generate the ylide. In exp. 076 the preformed ylide solution was added to a DMSO solution of farnesyl acetone (1 vol.). In exp. 077 reverse addition was tested—the solution of farnesyl acetone was added to the ylide solution. No major difference was observed: both reactions reached high conversion and gave high chemoselectivity after being stirred 20 h (94.9%-a/a epoxide for exp. 076 vs. 96.0%-a/a for exp. 077).

In all following experiments the ketone was added to the preformed ylide solution.

Experiments 078 and 079. The ylide was also generated with KOtBu but in THF and MeTHF respectively and at 65° C. IT. After 5 h preformation time, the reaction mixture was cooled down to 25° C. and farnesyl acetone was added. Despite that the starting material was fully converted in the two reactions, chemoselectivities were significantly lower (88.7%-a/a epoxide for exp. 078 and 83.9%-a/a for exp. 079).

Experiments 080-083. Four reactions were additionally conducted with the KOtBu/DMSO system. Trimethylsulfoxonium chloride was swapped in all reactions by the iodine salt analogue. In exp. 080, a solution of famesyl acetone solution (in DMSO) (1 vol.) was added to the reaction mixture once the ylide was preformed 1 h at 25° C. in DMSO (3.2 vol./Farnesyl acetone). In exp. 081 and 082 the addition of farnesyl acetone to the ylide solution was achieved neat at room temperature or neat at 0° C. respectively. In exp. 083, a 1:1 solution of MeTHF/DMSO was used to pre-form the ylide as an attempt to reduce the amount of DMSO and facilitate the work-up of the reaction. All four approaches gave promising results, with high conversion of Farnesyl acetone (<0.6%-a/a starting material detected by GC) and high chemoselectivity (>96.4%-a/a). For experiments 080, 081, and 082, once stirring was stopped after 20 h reaction time, two phases were spontaneously formed without any additional quench.

Separation of the two phases of exp. 081 followed by GC and HPLC analyses showed that the product decanted from DMSO and could be readily isolated, removing the bulk of the polar solvent. In addition the product was found to contain low amounts of DMSO and the organic phase contained low amounts of Farnesyl epoxide. ¹H NMR analysis further confirmed that the epoxide was isolated with high purity. Only the presence of tBuOH (0.24 eq./product, ratio determined by ¹H NMR) and DMSO (0.56 eq./product, ratio determined by ¹H NMR) was found. No other impurities were visible by ¹H NMR (300 Mhz).

Experiment 084. The reaction was scaled-up, duplicating the conditions of exp. 081. As expected, complete conversion of Farnesyl acetone was confirmed (97.5%-a/a chemoselectivity by GC vs. 95.8%-a/a in exp. 081), and decantation (removal of the bottom DMSO layer) of the reaction mixture allowed rapid isolation of the crude epoxide while removing the bulk of DMSO (28 g crude isolated=104% yield). The product was again found to contain approx. 0.5 eq. DMSO and 0.25 eq. tBuOH, so a work-up procedure was developed.

The first attempts at dissolving the crude epoxide in 10 vol. heptane, toluene, TBME or MeTHF followed by wash with either brine, 1:1 half. sat. NaCl or water were unsuccessful as all biphasic mixtures generated emulsion upon shaking which could not be separated. iPrOAc gave promising results when combined with 1:1 half sat. NaCl. It was possible to increase the concentration using 6 vs. 10 vol. of the solvent and 2.5 vol. of 1:1 half sat. NaCl (/mass crude). 3 washes were applied followed by one final wash with 5 vol. of brine and the resulting solution was eventually dried on 0.5 wt (/mass crude) of Na₂SO₄. Upon concentration on a rotavapor at 45° C. for 1 h, 22.5 g of product (85% yield) was obtained with 96.3% purity determined by GC and 98.0% purity by qNMR. 1.5%-wt/wt of iPrOAc could be integrated, giving a corrected purity of 99.5%.

The worked-up product was then purified by short path distillation. Prior to conducting the distillation, a sample of the epoxide was heated neat at 150° C. in a vial under air as stress test for 30 minutes. No decomposition was observed by ¹H NMR (and no formation of polymer could be visually detected either). Based on the boiling point of Farnesyl acetone found in literature (bp=335° C. at 760 mmHg), the boiling point of Farnesyl epoxide was estimated at approx. 115° C. under a reduced pressure of 0.15 mbar at which the distillation was conducted. Thus, the temperature was set to 135° C. to ensure complete distillation of the product. Overall, 17 g product was recovered (65% yield) but it is reasonable to assume that a fraction of the product was lost stuck in the apparatus, as only 1.9 g of residue was collected. The purity of the distilled product was measured by qNMR as 99.1% and by GC as 96.6% (vs. 99.5% by qNMR and 96.3% by GC for the corresponding worked-up crude).

Experiment 087. The reaction was scaled-up once again on 25 g scale and the work-up was applied once the solvent was separated from the product by decantation. 22.2 g of worked-up crude was obtained vs. 22.5 g in the analogous exp. 084, which highlights the reproducibly of the developed conditions. The oxirane was obtained with 96.1% purity by qNMR but the sample contained 2.6% iPrOAc, so the purity was corrected to 98.7%. GC analysis indicated 94.2% purity.

Prior to concentration, two 10 ml samples of the iPrOAc solution containing an estimated gram of the product were filtered on 5 wt (/mass product) SiO₂ and neutral alumina. Upon concentration, both samples were analyzed by GC and qNMR to compare their purity.

The purity of the SiO₂ filtered sample was determined as 99.8% by qNMR and 97.2% by GC. The purity of the Alumina filtered sample was determined as 96.2% by qNMR and 95.4% by GC.

### Conclusion

The synthesis of the epoxide was optimized and process friendly conditions were successfully developed. The reaction can be performed in only 3.2 volumes of DMSO with 1.2 eq. of the ylide and 1.15 eq. of the base at room temperature for 18 h. DMSO was found to decant from the Farnesyl epoxide once the reaction was complete, resulting in simple separation of the two phases and affording the crude product with a relatively high initial purity. Remaining traces of DMSO (0.56 eq.) and tBuOH (0.24 eq.) could be easily removed by simple work-up, furnishing the oxirane with 85-86% yield and >98% purity (qNMR).

### Example 7A. Alternative Synthesis of RAC-EE-2

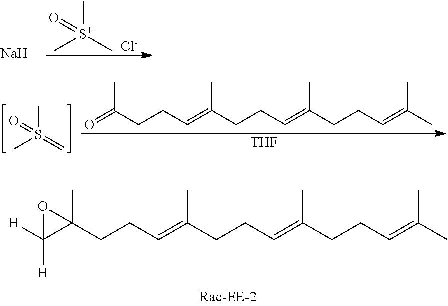

Racemic Farnesyl epoxide Rac-EE-2 was prepared using the following conditions.

| Exp. No. | Solvent | | T | t | Conv. | Sel. | Isolated | |
|---|---|---|---|---|---|---|---|---|
| (Scale) | (volumes) | Conditions | [° C.] | [h] | [%] | [%] | yield[%] | Comment(s) |
| 001 (950 mg) | THF (7) | Trimethylsulfoxonium chloride/NaH | 25 | 17 | >99 | >99 | 85 | 850 mg isolated |

Dimethylsulfoxonium methylide was generated in situ. The mineral oil of sodium hydride mineral dispersion was removed by petroleum ether/decanting procedure giving a suspension of NaH in petroleum ether under argon.

After refilling with Ar, trimethylsulfoxonium chloride and dry THF were added. With stirring, the mixture was heated to reflux in an oil bath. The evolution of hydrogen gas was fairly rapid at first, but after several minutes it ceased. After approximately 2 h, rapid hydrogen evolution again began and the reaction was finished as was evidenced by the lack of hydrogen evolution. After refluxing for 5 h, the reaction was allowed to cooled to rt.

Farnesyl acetone (950 mg, 3.62 mmol, 0.8 equiv.) was added to the reaction mixture. The reaction was allowed to be stirred for 17 h. The reaction was monitored by TLC (EtOAc/heptane 5:95) using phosphomolybdic acid as staining agent. Additionally, the reaction was monitored by achiral HPLC (Ascentis express phenyl hexyl column, see HPLC Method 1 described below). The reaction was quenched after 99% conversion of starting material. After concentration in vacuo, the resulting residue was purified by silica gel chromatography (EtOAc/n-hexane=5:95) to give 850 mg (85%).

Analytical separation of the epoxide enantiomers was achieved using HPLC on AD-H column (eluting with heptane/EtOH/MeOH (99.75:0.125:0.125)). Enantiomer 1 eluted at 7.7 minutes, and enantiomer 2 eluted at 8.7 minutes. The two enantiomer peaks were not completely separate at baseline.

The following HPLC Methods 1 to 4 illustrate the analysis techniques for the subsequent examples.

### HPLC Analytical Method 1 (Achiral, Acidic)

The following standard analytical HPLC method for the determination of the conversion and chemoselectivity of specified reactions.
Software: Agilent Series 1100
Column: ASCENTIS EXPRESS PHENYL HEXYL CAT50483-U/15×4.6 mm, 5 μm/USOK001365
Eluent: A: H₂O with 0.1% vol/vol TFA
   B: MeCN with 0.1% vol/vol TFA

Gradient:

| t (min) | % B | flow (mL/min) |
|---|---|---|
| 0 | 15 | 1 |
| 0.5 | 15 | 1 |
| 7.5 | 100 | 1 |
| 11.5 | 100 | 1 |
| 12.5 | 15 | 1 |

Detector,λ: UV-Detector λ=254 nm, 216 nm, 210 nm, 230 nm, 280 nm

Inject. Vol.: 2 μL
Temperature: 30° C.
Retention times:

| | |
|---|---|
| Farnesyl acetone | 7.9 min |
| Epoxide EE-2 | 8.1 min |

### HPLC Method 2 (Achiral, Basic)

The following analytical HPLC was used to monitor the epoxide coupling step (conversion and chemoselectivity).
Software: Agilent Series 1100
Column: XBridge BEH C18 2.5 um (3.0×75 mm)
Eluent: —A: 5 mM ammonium formate buffer, pH 9
   —B: MeCN

Gradient:

| t (min) | % B | flow (mL/min) |
|---|---|---|
| 0 | 60 | 0.8 |
| 5.0 | 95 | 0.8 |
| 14.0 | 95 | 0.8 |
| 19.0 | 60 | 0.8 |

Detector,λ: UV-Detector λ=254 nm, 216 nm, 220
Inject. Vol.: 2 μL
Temperature: 40° C.
Retention times:

| | |
|---|---|
| Farnesyl acetone | 4.3 min |
| Epoxide EE-2 | 5.2 min |

### Method 3 (Chiral Organic)

The following analytical chiral HPLC method for the determination of the enantioselectivity of specified reactions.
Software: Agilent Series 1100
Column: Chiralpak AD-H/4.6×250 mm/FA04
Eluent: A: Heptane (99.75%)
   B: EtOH (0.75%)
   Isocratic
Detector,λ: UV-Detector λ=254 nm, 220 nm (216 to be implemented).
Inject. Vol.: 2 μL
Temperature: 30° C.
Flow: 1 mL/min
Retention times:

| | |
|---|---|
| Farnesyl acetone | 5.1 min |
| EE-2 first eluting enantiomer | 5.4 min |
| EE-2 second eluting enantiomer | 6.5 min |

### Method 4 (Chiral Reverse Phase)

The following analytical chiral HPLC method for the determination of the enantioselectivity of specified reactions.
Software: Agilent Series 1100
Column: Chiralpak AD-RH/4.6×150 mm, 5 μm/LHS6230
Eluent: A: H₂O (90%)
   B: MeOH (10%)
   Isocratic
Detector: UV-Detector λ=254 nm, 216 nm, 210 nm.
Inject. Vol.: 2 μL
Temperature: 35° C.
Flow: 1 mL/min
Retention times:

| | |
|---|---|
| Farnesyl acetone | 6.9 min |
| EE-2 first eluting enantiomer | 13.9 min |
| EE-2 second eluting enantiomer | 20.6 min |

### Example 9. Preparation of Enantioenriched Farnesyl Epoxide ((R)-EE-2)

Provided herein are methods of producing enantioenriched farnesyl epoxide ((R)-EE-2), using an approach (Scheme A). Direct asymmetric Corey-Chaykovsky epoxidation of farnesyl acetone in the presence of catalyst (R-LLB) and additive (TMPPO) gave farnesyl epoxide (R)-EE-2 (91.2% ee). Detailed studies of the approach is discussed herein.

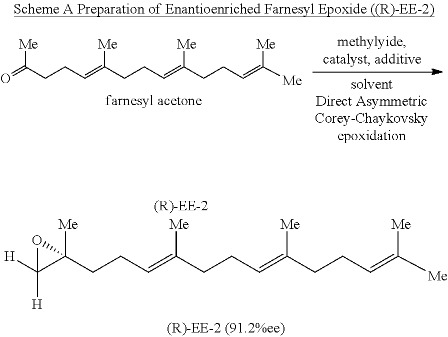 [SMILES: CC(=O)CC/C=C(\C)CC/C=C(\C)CCC=C(C)C ; CC(C)=CCC/C(C)=C/CC/C(C)=C/CC[C@]1(C)CO1]

### Asymmetric Corey-Chaykovsky Epoxidation

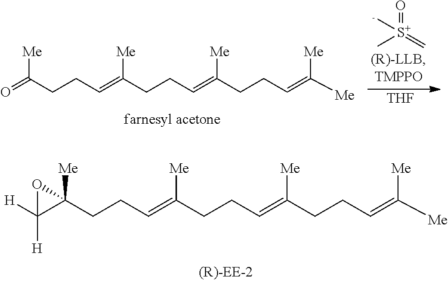

The chiral epoxide (R)-EE-2 was prepared via catalytic asymmetric epoxidation.

### Steps 1-2: Preparation of Chiral BINOL-Based Lanthanum Complex (R)-LLB and TMPPO

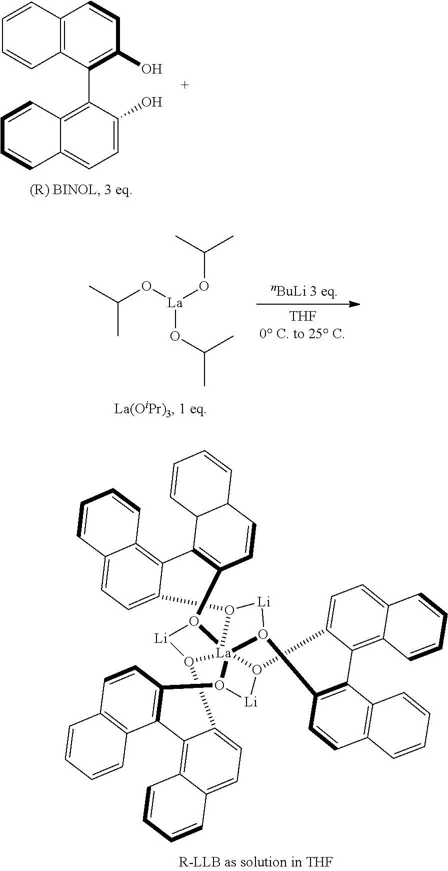

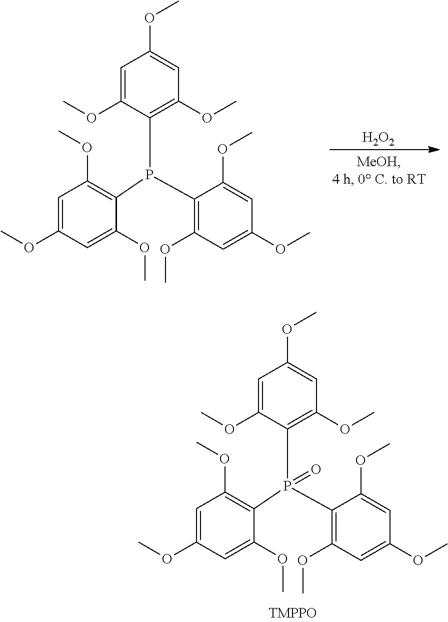

The syntheses of the catalysts (R)-La-Li₃-(binaphthoxide)₃ ((R)-LLB) and of tris(2,6-dimethoxyphenyl)phosphine oxide (TMPPO) were conducted according to the reference Sone, T.; Yamaguchi, A.; Matsunaga, S.; Shibasaki, M. J. Am. Chem. Soc. 2008, 130, 10078. No particular issues were noted. Crude TMPPO was isolated with 75% yield. The crude product was recrystallized (AcOEt/DCM) to afford the phosphine oxide (3.95, g 78%), and >99% purity (HPLC). The recrystallization was performed by dissolving the crude material (9 g) in 10-15 mL of DCM. AcOEt (100 mL) was added slowly and the product precipitated. Then DCM (and partially AcOEt) were removed in vacuo and the product was filtered and dried.

### Step 3: Preparation of Dimethylsulfonium Methylide

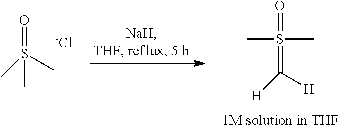

The synthesis of the dimethylsulfoxonium methylide was conducted as described by Corey, E. J.: Chaykovsky, M. J. Am. Chem. Soc. 1965, 87, 1353.

### Step 4: Preparation of Enantioenriched Farnesyl Epoxide ((R)-EE-2)

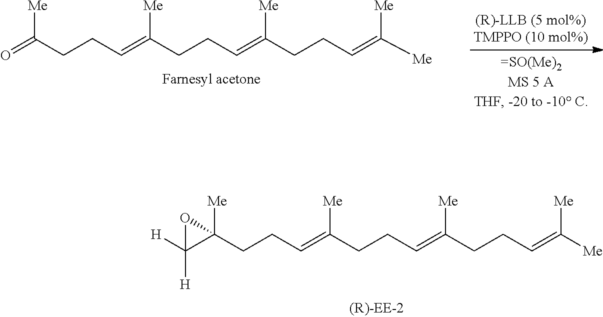 [SMILES: CC(=O)CC/C=C(\C)CC/C=C(\C)CCC=C(C)C ; CC(C)=CCC/C(C)=C/CC/C(C)=C/CC[C@]1(C)CO1]

5 Å molecular sieves powder (16 g) were activated at 200° C. under reduced pressure (ca. 1 mm Hg) for 15 h. After backfilling with argon and cooling to room temperature, TMPPO (2.09 g, 3.8 mmol, 10 mol %), (R)-LLB (0.1 M in THE solution, 19 mL, 1.9 mmol; 5 mol %) and THF (320 mL, 0.12 M) were added at room temperature. After being stirred for 30 min at room temperature, dimethylsulfoxonium methylide (45.7 mL, 45.7 mmol, 1 M in THF, 1.2 eq) was added to the mixture. The reaction was allowed to stir for 30 min at the same temperature. The reaction was cooled to −20° C. and farnesyl acetone (10 g, 38.1 mmol, 1 eq.) was added. The reaction temperature was slowly raised to −10° C. and stirred at this temperature for 18 h. The reaction was monitored by chiral HPLC using HPLC Method 1. After 18 h, HPLC analyses showed 88% conversion. The HPLC analysis also showed clean reaction with no byproducts. Selectivity was 99% and enantioselectivity was 91% ee.

Additional dimethyloxosulfonium methylide (0.2 eq.) was added, and the reaction allowed to stir for 4 h. No additional conversion was observed, and selectivity and enantioselectivity were also not affected.

NH₄Cl aq. and iPrOAc were added to quench the reaction. The water layer was extracted with iPrOAc (200 mL, ×2). The combined organic layers were washed with brine, and dried over Na₂SO₄. After evaporating the solvent under reduced pressure, the residue was purified by flash column chromatography (neutral approximately 1 kg SiO₂, and a mixture of heptane/EtOAc: 95:5 as the eluent) to give product (enantiomer fraction 1 and enantiomer fraction 2).

THF can be substituted with alternative solvents including toluene, MeTHF, and CPME to avoid use of it which might be problematic upon scale-up.

### Step 5. Further Purification to Isolate Pure Epoxide

Isolated Fraction 1 (7.3 g, 69%) was collected with 99% purity determined by HPLC (1%-a/a farnesyl acetone could be integrated) and 89% purity determined by qNMR (lower purity exclusively due to the presence of heptane clearly visible on the ¹H spectrum, no additional byproducts were visible).

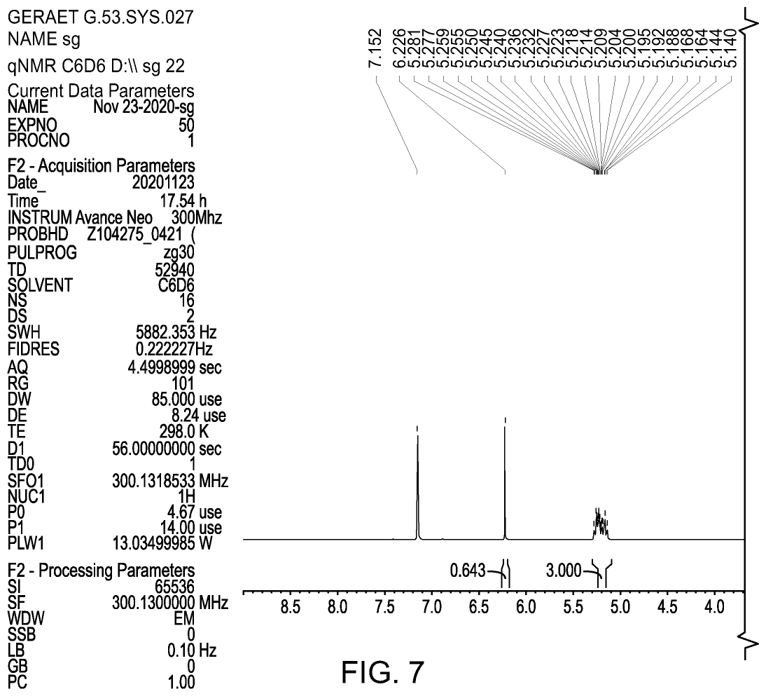

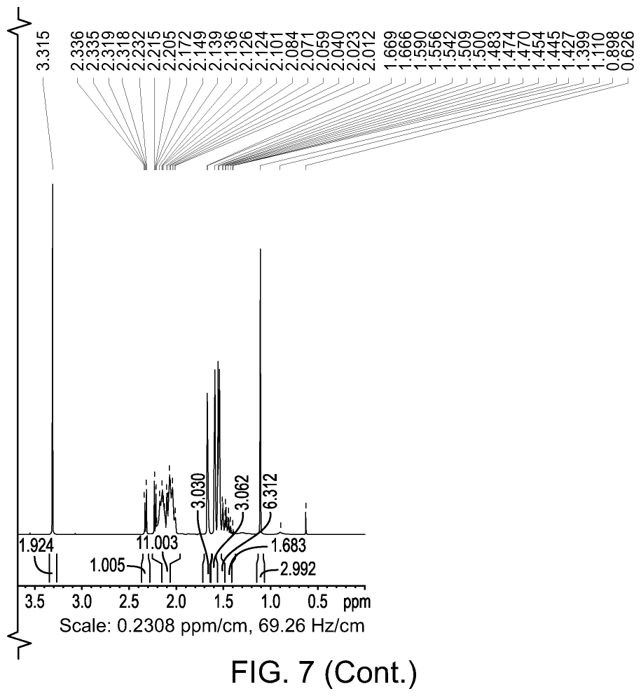

Isolated Fraction 2 (2.6 g) was separately isolated. The product included more (4%-a/a) farnesyl acetone than in the fractions for Isolated Fraction 1. Consequently, the Isolated Fraction 2 was chromatographed again to give product (2.5 g, 24%) with >99% purity by HPLC (no trace of famesyl acetone 1 was detected) and 97.5% purity by qNMR (trace of heptane identified). See FIG. 7.

Both epoxide (Enantiomers 1 and 2) fractions were analyzed by chiral HPLC. The Isolated Fraction 1 was found to have 92.0% ee and Isolated Fraction 2 was found to have 91.5% ee with the same stereoisomer being the predominant one. The slight difference of enantiomeric excess could be explained by the broadness of the peaks, resulting in imprecise integration.

Overall, considering both Isolated Fraction 1 and Isolated Fraction 2 and their respective purity, the yield of the reaction was 86%. The fractions were not combined. Example 10.

The mixed catalyst was prepared according to the following scheme.

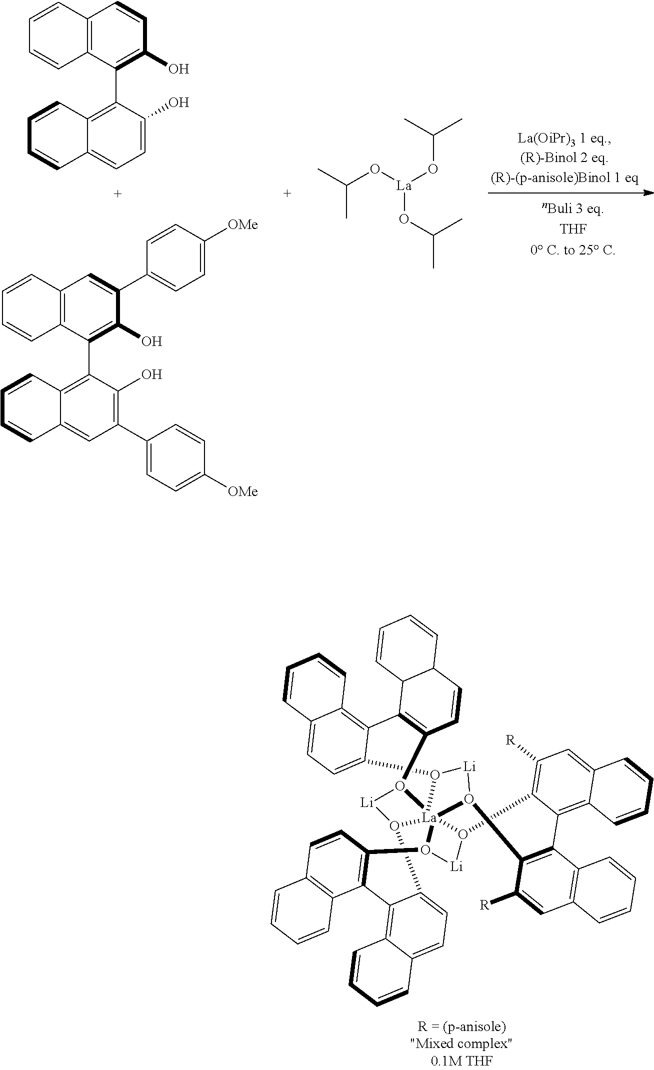

Binol (453 mg, 2 eq.) was used in combination with one equivalent of the 3,3′-(p-anisole) functionalized derivative (394 mg, 1 eq.) to prepare the Mixed Complex catalyst. No analytical assessment on the isolated complex was conducted, and the structure is illustrated based on the applied reagents.

R-EE-2 was prepared using Mixed Complex catalyst according to the following scheme.

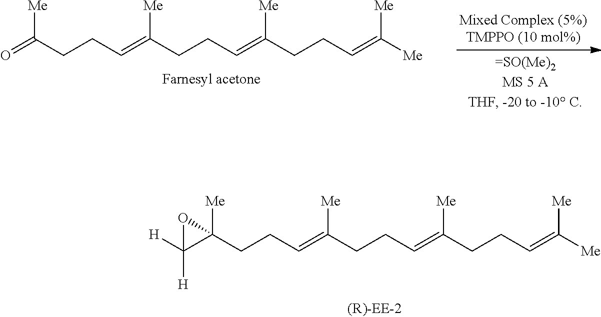 [SMILES: CC(=O)CC/C=C(\C)CC/C=C(\C)CCC=C(C)C ; CC(C)=CCC/C(C)=C/CC/C(C)=C/CC[C@]1(C)CO1]

Farnesyl acetone (210 mg, 0.8 mmol, 1 eq.), dimethylsulfoxonium methylide (1 M solution in THF, 0.960 mL, 0.960 mmol, 1.2 eq.), Mixed Complex (0.1 M in THE solution, 0.4 mL, 0.04 mmol: 5 mol %), TMPPO (43 mg, 10 mol %), 5 Å molecular sieves (400 mg) were added to THF (8 mL, 0.1M). T=−20 to −10° C. The reaction was allowed to proceed for 18 h.

The conversion to R-EE-2 and chemoselectivity of R-EE-2 were determined by achiral HPLC at 216 nm using method 2. The enantioselectivity was determined by chiral HPLC at 216 nm (see analytical method 3, section Error! Reference source not found.). Uncorrected integrals were used. Mixed complex (2 Binol units, 1 p-anisole Binol unit) gave moderate conversion (56%) and good enantioselectivity (76%) compared the lanthanum complex built with p-anisole Binol units only. Compared to the results in Example 9, the increasing steric demand of Mixed Complex was not beneficial to the reaction and resulted in erosion of enantiomeric excess.

The disclosures of all publications, patents, patent applications and published patent applications referred to herein by an identifying citation are hereby incorporated herein by reference in their entirety.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

1. A compound of the formula: 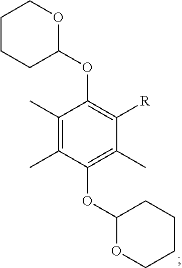
wherein R is —CH₃ or 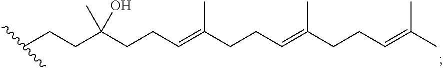 [SMILES: *CCC(C)(O)CC/C=C(\C)CC/C=C(\C)CCC=C(C)C]
or a salt thereof; and/or an isomer thereof;
and/or a solvate or a hydrate thereof.

2. The compound of claim 1, wherein R is: 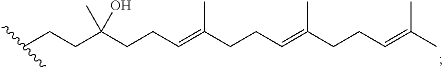 [SMILES: *CCC(C)(O)CC/C=C(\C)CC/C=C(\C)CCC=C(C)C]
or a salt thereof; and/or an isomer thereof;
and/or a solvate or a hydrate thereof.

3. The compound of claim 1, wherein R is: 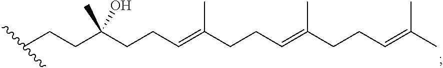 [SMILES: *CC[C@](C)(O)CC/C=C(\C)CC/C=C(\C)CCC=C(C)C]
or a salt thereof;
and/or a solvate or a hydrate thereof.

4. The compound of claim 1, wherein R is: 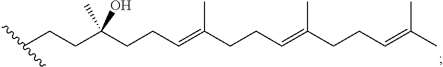 [SMILES: *CC[C@@](C)(O)CC/C=C(\C)CC/C=C(\C)CCC=C(C)C]
or a salt thereof;
and/or a solvate or a hydrate thereof.

5. The compound of claim 3, wherein R is: 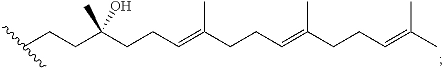 [SMILES: *CC[C@](C)(O)CC/C=C(\C)CC/C=C(\C)CCC=C(C)C]
and the compound is the E-isomer at the 6 and 10-positions;
or a salt thereof;
and/or a solvate or a hydrate thereof.

6. The compound of claim 1, wherein R is —CH₃; or a salt thereof; and/or an isomer thereof; and/or a solvate or a hydrate thereof.

7. The compound of claim 1, wherein the compound is a lithium salt thereof.

8. The compound of claim 1, wherein the compound is not a solvate and is not a hydrate thereof.

9. A compound of the formula: 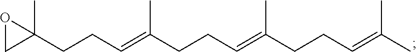 [SMILES: CC(C)=CCC/C(C)=C/CC/C(C)=C/CCC1(C)CO1]
or a salt thereof; and/or a solvate or hydrate thereof.

10. The compound of claim 9, wherein the compound has the formula: 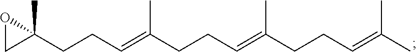 [SMILES: CC(C)=CCC/C(C)=C/CC/C(C)=C/CC[C@]1(C)CO1]
or a salt thereof; and/or a solvate or hydrate thereof.

11. The compound of claim 10, wherein the compound has the formula: 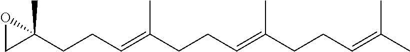 [SMILES: CC(C)=CCC/C(C)=C/CC/C(C)=C/CC[C@]1(C)CO1]
and the compound is the E-isomer at the 3 and 7-positions; or a salt thereof; and/or a solvate or hydrate thereof.

12. The compound of claim 9, wherein the compound has the formula: 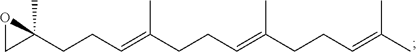 [SMILES: CC(C)=CCC/C(C)=C/CC/C(C)=C/CC[C@@]1(C)CO1]
or a salt thereof; and/or a solvate or hydrate thereof.

13. The compound of claim 9, wherein the compound is not a solvate and is not a hydrate thereof.

14-79. (canceled)

80. The compound of claim 10, wherein the compound is not a solvate and is not a hydrate thereof.

81. The compound of claim 11, wherein the compound is not a solvate and is not a hydrate thereof.
